(19) United States
(12) Patent Application Publication
(10) Pub. No.: US 2013/0045226 A1
(43) Pub. Date: Feb. 21, 2013

(54) METHOD FOR FORMING AND EXTRACTING SOLID PELLETS COMPRISING OIL-CONTAINING MICROBES

(71) Applicants: Marios Avgousti, Kennett Square, PA (US); Timothy Allan Bell, Wilmington, DE (US); Oliver Walter Gutsche, Wilmington, DE (US); Keith W. Hutchenson, Lincoln University, PA (US); Robert D. Orlandi, Landenberg, PA (US)

(72) Inventors: Marios Avgousti, Kennett Square, PA (US); Timothy Allan Bell, Wilmington, DE (US); Oliver Walter Gutsche, Wilmington, DE (US); Keith W. Hutchenson, Lincoln University, PA (US); Robert D. Orlandi, Landenberg, PA (US)

(73) Assignee: E I DU PONT DE NEMOURS AND COMPANY, Wilmington, DE (US)

(21) Appl. No.: 13/370,597

(22) Filed: Feb. 10, 2012

Related U.S. Application Data
(60) Provisional application No. 61/441,836, filed on Feb. 11, 2011.

(51) Int. Cl. B29C 47/08; B02C 23/00; A23D 9/05; A61K 35/66; A61K 35/74

(52) U.S. Cl. 424/195.16; 424/780; 426/601; 29/527.1; 264/210.1

(57) ABSTRACT

A process including: (a) mixing a microbial biomass, comprising oil-containing microbes, and at least one grinding agent capable of absorbing oil, to provide a disrupted biomass mix; (b) blending at least one binding agent with said disrupted biomass mix to provide a fixable mix capable of forming a solid pellet; and, (c) forming the solid pellet from the fixable mix. The process optionally includes extracting the solid pellet with a solvent to provide an extracted microbial oil.

This application claims the benefit of U.S. Provisional Application No. 61/441,836, filed Feb. 11, 2011, which is hereby incorporated by reference in its entirety.

### FIELD OF THE INVENTION

The present invention relates to a method for forming solid pellets from microbial biomass comprising oil-containing microbes and a method for extracting said solid pellets to provide oil.

### BACKGROUND OF THE INVENTION

There has been growing interest in including PUFAs such as eicosapentaenoic acid (EPA; omega-3) and docosahexaenoic acid (DHA; omega-3) in pharmaceutical and dietary products. Polyunsaturated fatty acid (PUFA)-containing lipid compositions can be obtained, for example, from natural microbial sources, from recombinant microorganisms, or from fish oil and marine plankton. PUFA-containing lipid compositions are recognized as being oxidatively unstable under certain conditions, which necessitates expending considerable care to obtain un-oxidized compositions.

U.S. Pat. No. 6,727,373 discloses a microbial PUFA-containing oil with a high triglyceride content and a high oxidative stability. In addition, a method is described for the recovery of such oil from a microbial biomass derived from a pasteurized fermentation broth, wherein the microbial biomass is subjected to extrusion to form granular particles, dried, and the oil is then extracted from the dried granules using an appropriate solvent.

U.S. Pat. No. 6,258,964 discloses a method of extracting liposoluble components contained in microbial cells, wherein the method requires drying microbial cells containing liposoluble components, simultaneously disrupting and molding the dried microbial cells into pellets by use of an extruder, and extracting the contained liposoluble components by use of an organic solvent.

U.S. Pat. Appl. Pub. No. 2009/0227678 discloses a process for obtaining lipid from a composition comprising cells and water, the process comprising contacting the composition with a desiccant, and recovering the lipid from the cells.

A process flow diagram developed for a continuous countercurrent supercritical carbon dioxide fractionation process that produces high concentration EPA is disclosed by V. J. Krukonis et al. (Adv. Seafood Biochem., Pap. Am. Chem. Soc. Annu. Meet. (1992), Meeting Date 1987, 169-79).

Methods for efficient recovery of oil from microbial biomass are desired.

### SUMMARY OF INVENTION

In a first embodiment, the invention concerns a process comprising:
a) mixing a microbial biomass, having a moisture level and comprising oil-containing microbes, and at least one grinding agent capable of absorbing oil, to provide a disrupted biomass mix;
b) blending at least one binding agent with said disrupted biomass mix to provide a fixable mix capable of forming a solid pellet; and,
c) forming said solid pellet from the fixable mix.

In a second embodiment of the process, the moisture level of the microbial biomass is preferably in the range of about 1 to 10 weight percent.

In a third embodiment of the process, the at least one grinding agent preferably has a property selected from the group consisting of:
a) said at least one grinding agent is a particle having a Moh hardness of 2.0 to 6.0 and an oil absorption coefficient of 0.8 or higher as determined according to ASTM Method D1483-60;
b) said at least one grinding agent is selected from the group consisting of silica and silicate; and,
c) said at least one grinding agent is present at about 1 to 20 weight percent, based on the summation of the weight of microbial biomass, grinding agent and binding agent in the solid pellet.

In a fourth embodiment of the process, the at least one binding agent is preferably has a property selected from the group consisting of:
a) said at least one binding agent is selected from water and carbohydrates selected from the group consisting of: sucrose, lactose, fructose, glucose, and soluble starch; and,
b) said at least one binding agent is present at about 0.5 to 10 weight percent, based on the summation of the weight of microbial biomass, grinding agent and binding agent in the solid pellet.

In a fifth embodiment of the process, steps (a) mixing said biomass and (b) blending at least one binding agent are performed in an extruder, are performed simultaneously, or are performed simultaneously in an extruder.

In a sixth embodiment of the process, step (c) forming said solid pellet from said fixable mix comprises a step selected from the group consisting of:
(i) extruding said fixable mix through a die to form strands;
(ii) drying and breaking said strands; and,
(iii) combinations of step (i) extruding said fixable mix through a die to form strands and step (ii) drying and breaking said strands.

In a seventh embodiment of the process, the pellets are formed using a granulator, are dried using a fluid bed dryer, or are formed using a granulator and are dried using a fluid bed dryer.

In a eighth embodiment of the process, the oil-containing microbes are selected from the group consisting of yeast, algae, fungi, bacteria, euglenoids, stramenopiles and oomycetes. Preferably, the oil-containing microbes comprise at least one polyunsaturated fatty acid in the oil.

In a ninth embodiment of the process, the microbial biomass is a disrupted biomass, having a disruption efficiency of at least 50% of the oil-containing microbes.

In a tenth embodiment of the process, the microbial biomass is disrupted to produce a disrupted biomass in a twin screw extruder comprising:
(a) a total specific energy input (SEI) of about 0.04 to 0.4 KW/(kg/hr);
(b) compaction zone using bushing elements with progressively shorter pitch length; and,
(c) a compression zone using flow restriction;
   wherein the compaction zone is prior to the compression zone within the extruder.

The flow restriction is preferably provided by reverse screw elements, restriction/blister ring elements or kneading elements.

In an eleventh embodiment of the process, wherein the microbial biomass is a disrupted biomass, having a disruption efficiency of at least 50% of the oil-containing microbes, the process may further comprise step (d), extracting the solid pellet with a solvent to provide an extract comprising the oil.

Preferably, the solvent comprises liquid or supercritical fluid carbon dioxide.

In an eleventh embodiment of the process is the pelletized oil-containing microbial biomass made therefrom.

In a twelfth embodiment is a solid pellet comprising
a) about 70 to about 98.5 weight percent of disrupted biomass comprising oil-containing microbes;
b) about 1 to about 20 weight percent of at least one grinding agent capable of absorbing oil; and,
c) about 0.5 to 10 weight percent of at least one binding agent;
wherein the weight percents of (a), (b) and (c) are based on the summation of (a), (b) and (c) in the solid pellet.
   The solid pellets preferably have an average diameter of about 0.5 to about 1.5 mm and an average length of about 2.0 to about 8.0 mm. Preferably, solid pellets have a moisture level of about 0.1 to 5.0 weight percent.

### DESCRIPTION OF FIGURE

FIG. 1 illustrates a custom high-pressure extraction apparatus flowsheet.

### BIOLOGICAL DEPOSITS

The following biological materials have been deposited with the American Type Culture Collection (ATCC), 10801 University Boulevard, Manassas, Va. 20110-2209, and bear the following designations, accession numbers and dates of deposit.

| Biological Material | Accession No. | Date of Deposit |
|---|---|---|
| Yarrowia lipolytica Y8412 | ATCC PTA-10026 | May 14, 2009 |
| Yarrowia lipolytica Y8259 | ATCC PTA-10027 | May 14, 2009 |

The biological materials listed above were deposited under the terms of the Budapest Treaty on the International Recognition of the Deposit of Microorganisms for the Purposes of Patent Procedure. The listed deposit will be maintained in the indicated international depository for at least 30 years and will be made available to the public upon the grant of a patent disclosing it. The availability of a deposit does not constitute a license to practice the subject invention in derogation of patent rights granted by government action.

Yarrowia lipolytica Y9502 was derived from Y. lipolytica Y8412, according to the methodology described in U.S. Pat. Appl. Pub. No. 2010-0317072-A1. Similarly, Yarrowia lipolytica Y8672 was derived from Y. lipolytica Y8259, according to the methodology described in U.S. Pat. Appl. Pub. No. 2010-0317072-A1.

### DETAILED DESCRIPTION OF THE INVENTION

The disclosures of all patent and non-patent literature cited herein are hereby incorporated by reference in their entireties.

When an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

As used herein, the terms “comprises”, “comprising”, “includes”, “including”, “has”, “having”, “contains” or “containing”, or any other variation thereof, are intended to cover a non-exclusive inclusion. For example, a composition, mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e., occurrences) of the element or component. Therefore, “a” or “an” should be read to include one or at least one, and the singular word form of the element or component also includes the plural unless the number is obviously meant to be singular.

As used herein the term “invention” or “present invention” is intended to refer to all aspects and embodiments of the invention as described in the claims and specification herein and should not be read so as to be limited to any particular embodiment or aspect.

The following definitions are used in this disclosure:

“Carbon dioxide” is abbreviated as “CO₂”.

“American Type Culture Collection” is abbreviated as “ATCC”.

“Polyunsaturated fatty acid(s)” is abbreviated as “PUFA(s)”.

“Phospholipids” are abbreviated as “PLs”.

“Monoacylglycerols” are abbreviated as “MAGs”.

“Diacylglycerols” are abbreviated as “DAGs”.

“Triacylglycerols” are abbreviated as “TAGs”. Herein the term “triacylglycerols” (TAGs) is synonymous with the term “triacylglycerides” and refers to neutral lipids composed of three fatty acyl residues esterified to a glycerol molecule. TAGs can contain long chain PUFAs and saturated fatty acids, as well as shorter chain saturated and unsaturated fatty acids.

“Free fatty acids” are abbreviated as “FFAs”.

“Total fatty acids” are abbreviated as “TFAs”.

“Fatty acid methyl esters” are abbreviated as “FAMEs”.

“Dry cell weight” is abbreviated as “DCW”.

As used herein the term “microbial biomass” refers to microbial cellular material from a microbial fermentation of oil-containing microbes, conducted to produce microbial oil. The microbial biomass may be in the form of whole cells, whole cell lysates, homogenized cells, partially hydrolyzed cellular material, and/or disrupted cells. Preferably, the microbial oil comprises at least one PUFA.

The term “untreated microbial biomass” refers to microbial biomass prior to extraction with a solvent. Optionally, untreated microbial biomass may be subjected to at least one mechanical process (e.g., by drying the biomass, disrupting the biomass, pelletizing the biomass, or a combination of these) prior to extraction with a solvent.

The term “disrupted microbial biomass” refers to microbial biomass that has been subjected to a process of disruption, wherein said disruption results in a disruption efficiency of at least 50% of the microbial biomass.

The term “disruption efficiency” refers to the percent of cells walls that have been fractured or ruptured during processing, as determined qualitatively by optical visualization or as determined quantitatively according to the following formula: % disruption efficiency=(% free oil*100) divided by (% total oil), wherein % free oil and % total oil are measured for the solid pellet. Increased disruption efficiency of the microbial biomass typically leads to increased extraction yields of the microbial oil contained within the microbial biomass.

The term “percent total oil” refers to the total amount of all oil (e.g., including fatty acids from neutral lipid fractions [DAGs, MAGs, TAGs], free fatty acids, phospholipids, etc. present within cellular membranes, lipid bodies, etc.) that is present within a solid pellet sample. Percent total oil is effectively measured by converting all fatty acids within a pelletized sample that has been subjected to mechanical disruption, followed by methanolysis and methylation of acyl lipids. Thus, the sum of the fatty acids (expressed in triglyceride form) is taken to be the total oil content of the sample. In the present invention, percent total oil is preferentially determined by gently grinding a solid pellet into a fine powder using a mortar and pestle, and then weighing aliquots (in triplicate) for analysis. The fatty acids in the sample (existing primarily as triglycerides) are converted to the corresponding methyl esters by reaction with acetyl chloride/methanol at 80° C. A C15:0 internal standard is then added in known amounts to each sample for calibration purposes. Determination of the individual fatty acids is made by capillary gas chromatography with flame ionization detection (GC/FID). And, the sum of the fatty acids (expressed in triglyceride form) is taken to be the total oil content of the sample.

The term “percent free oil” refers to the amount of free and unbound oil (e.g., fatty acids expressed in triglyceride form, but not all phospholipids) that is readily available for extraction from a particular solid pellet sample. Thus, for example, an analysis of percent free oil will not include oil that is present in non-disrupted membrane-bound lipid bodies. In the present invention, percent free oil is preferentially determined by stirring a sample with n-heptane, centrifuging, and then evaporating the supernatant to dryness. The resulting residual oil is then determined gravimetrically and expressed as a weight percentage of the original sample.

The term “disrupted biomass mix” refers to the product obtained by mixing microbial biomass and at least one grinding agent.

The term “grinding agent” refers to an agent, capable of absorbing oil that is mixed with microbial biomass to yield disrupted biomass mix. Preferably, the at least one grinding agent is present at about 1 to 50 parts, based on 100 parts of microbial biomass. In some preferred embodiments, the grinding agent is a silica or silicate. Other preferred properties of the grinding agent are discussed infra.

The term “fixable mix” refers to the product obtained by blending at least one binding agent with disrupted biomass mix. The fixable mix is a mixture capable of forming a solid pellet upon removal of solvent (e.g., removal of water in a drying step).

The term “binding agent” refers to an agent that is blended with disrupted biomass mix to yield a fixable mix. Preferably, the at least one binding agent is present at about 0.5 to 20 parts, based on 100 parts of microbial biomass. In some preferred embodiments, the binding agent is a carbohydrate. Other preferred properties of the binding agent are discussed infra.

The term “solid pellet” refers to a pellet having structural rigidity and resistance to changes of shape or volume. Solid pellets are formed herein from microbial biomass via a process of “pelletization”. Typically, solid pellets have a final moisture level of about 0.1 to 5.0 weight percent, with a range about 0.5 to 3.0 weight percent more preferred.

As used herein the term “residual biomass” refers to microbial cellular material from a microbial fermentation that is conducted to produce microbial oil, which has been extracted at least once with a solvent (e.g., an inorganic or organic solvent). When the initial microbial biomass subjected to extraction is in the form of a solid pellet, the residual biomass may be referred to as a “residual pellet”.

The term “lipids” refer to any fat-soluble (i.e., lipophilic), naturally-occurring molecule. Lipids are a diverse group of compounds that have many key biological functions, such as structural components of cell membranes, energy storage sources and intermediates in signaling pathways. Lipids may be broadly defined as hydrophobic or amphiphilic small molecules that originate entirely or in part from either ketoacyl or isoprene groups. A general overview of lipids, based on the Lipid Metabolites and Pathways Strategy (LIPID MAPS) classification system (National Institute of General Medical Sciences, Bethesda, Md.), is shown below in Table 1.

TABLE 1
Overview of Lipid Classes

| Structural | | |
|---|---|---|
| Building | | |
| Block | Lipid Category | Examples Of Lipid Classes |
| Derived | Fatty Acyls | Includes fatty acids, eicosanoids, fatty |
| from | | esters and fatty amides |
| condensation | Glycerolipids | Includes mainly mono-, di- and tri- |
| of ketoacyl | | substituted glycerols, the most well-known |
| subunits | | being the fatty acid esters of glycerol |
| | | [“triacylglycerols”] |
| | Glycero- | Includes phosphatidylcholine, |
| | phospholipids | phosphatidylethanolamine, |
| | or | phosphatidylserine, phosphatidylinositols |
| | Phospholipids | and phosphatidic acids |
| | Sphingolipids | Includes ceramides, phospho-sphingolipids |
| | | (e.g., sphingomyelins), glycosphingolipids |
| | | (e.g., gangliosides), sphingosine, |
| | | cerebrosides |
| | Saccharolipids | Includes acylaminosugars, |
| | | acylamino-sugar glycans, acyltrehaloses, |
| | | acyltrehalose glycans |
| | Polyketides | Includes halogenated acetogenins, |
| | | polyenes, linear tetracyclines, |
| | | polyether antibiotics, flavonoids, |
| | | aromatic polyketides |
| Derived | Sterol Lipids | Includes sterols (e.g., cholesterol), C18 |
| from | | steroids (e.g., estrogens), C19 steroids |
| condensation | | (e.g., androgens), C21 steroids (e.g., |
| of isoprene | | progestogens, glucocorticoids and mineral- |
| subunits | | ocorticoids), secosteroids, bile acids |
| | Prenol Lipids | Includes isoprenoids, carotenoids, |
| | | quinones, hydroquinones, polyprenols, |
| | | hopanoids |

The term “oil” refers to a lipid substance that is liquid at 25° C. and usually polyunsaturated. In oleaginous organisms, oil constitutes a major part of the total lipid. “Oil” is composed primarily of triacylglycerols (TAGs) but may also contain other neutral lipids, phospholipids (PLs) and free fatty acids (FFAs). The fatty acid composition in the oil and the fatty acid composition of the total lipid are generally similar; thus, an increase or decrease in the concentration of PUFAs in the total lipid will correspond with an increase or decrease in the concentration of PUFAs in the oil, and vice versa.

“Neutral lipids” refer to those lipids commonly found in cells in lipid bodies as storage fats and are so called because at cellular pH, the lipids bear no charged groups. Generally, they are completely non-polar with no affinity for water. Neutral lipids generally refer to mono-, di-, and/or triesters of glycerol with fatty acids, also called monoacylglycerol, diacylglycerol or triacylglycerol (TAG), respectively, or collectively, acylglycerols. A hydrolysis reaction must occur to release FFAs from acylglycerols.

The term “extracted oil” refers to an oil that has been separated from cellular materials, such as the microorganism in which the oil was synthesized. Often, the amount of oil that may be extracted from the microorganism is proportional to the disruption efficiency.

Extracted oils are obtained through a wide variety of methods, the simplest of which involves physical means alone. For example, mechanical crushing using various press configurations (e.g., screw, expeller, piston, bead beaters, etc.) can separate oil from cellular materials. Alternatively, oil extraction can occur via treatment with various organic solvents (e.g., hexane, iso-hexane), enzymatic extraction, osmotic shock, ultrasonic extraction, supercritical fluid extraction (e.g., CO₂ extraction), saponification and combinations of these methods. Further purification or concentration of an extracted oil is optional.

The term “total fatty acids” (TFAs) herein refer to the sum of all cellular fatty acids that can be derivatized to fatty acid methyl esters (FAMEs) by the base transesterification method (as known in the art) in a given sample, which may be the biomass or oil, for example. Thus, total fatty acids include fatty acids from neutral lipid fractions (including DAGs, MAGs and TAGs) and from polar lipid fractions (including the phosphatidylcholine and the phosphatidylethanolamine fractions) but not FFAs.

The term “total lipid content” of cells is a measure of TFAs as a percent of the dry cell weight (DCW), although total lipid content can be approximated as a measure of FAMEs as a percent of the DCW (FAMEs % DCW). Thus, total lipid content (TFAs % DCW) is equivalent to, e.g., milligrams of total fatty acids per 100 milligrams of DCW.

The concentration of a fatty acid in the total lipid is expressed herein as a weight percent of TFAs (% TFAs), e.g., milligrams of the given fatty acid per 100 milligrams of TFAs. Unless otherwise specifically stated in the disclosure herein, reference to the percent of a given fatty acid with respect to total lipids is equivalent to concentration of the fatty acid as % TFAs (e.g., EPA of total lipids is equivalent to EPA % TFAs).

In some cases, it is useful to express the content of a given fatty acid(s) in a cell as its weight percent of the dry cell weight (% DCW). Thus, for example, eicosapentaenoic acid % DCW would be determined according to the following formula: (eicosapentaenoic acid % TFAs)*(TFAs DCW)]/100. The content of a given fatty acid(s) in a cell as its weight percent of the dry cell weight (% DCW) can be approximated, however, as: (eicosapentaenoic acid % TFAs)*(FAMEs % DCW)]/100.

The terms “lipid profile” and “lipid composition” are interchangeable and refer to the amount of individual fatty acids contained in a particular lipid fraction, such as in the total lipid or the oil, wherein the amount is expressed as a weight percent of TFAs. The sum of each individual fatty acid present in the mixture should be 100.

The term “fatty acids” refers to long chain aliphatic acids (alkanoic acids) of varying chain lengths, from about C₁₂ to C₂₂, although both longer and shorter chain-length acids are known. The predominant chain lengths are between C₁₆ and C₂₂. The structure of a fatty acid is represented by a simple notation system of “X:Y”, where X is the total number of carbon [“C”] atoms in the particular fatty acid and Y is the number of double bonds. Additional details concerning the differentiation between “saturated fatty acids” versus “unsaturated fatty acids”, “monounsaturated fatty acids” versus “polyunsaturated fatty acids” (PUFAs), and “omega-6 fatty acids” [“(ω-6” or “n-6”] versus “omega-3 fatty acids” [“ω-3” or “n-3”] are provided in U.S. Pat. No. 7,238,482, which is hereby incorporated herein by reference.

Nomenclature used to describe PUFAs herein is given in Table 2. In the column titled “Shorthand Notation”, the omega-reference system is used to indicate the number of carbons, the number of double bonds and the position of the double bond closest to the omega carbon, counting from the omega carbon, which is numbered 1 for this purpose. The remainder of the Table summarizes the common names of omega-3 and omega-6 fatty acids and their precursors, the abbreviations that will be used throughout the specification and the chemical name of each compound.

TABLE 2
Nomenclature of Polyunsaturated Fatty Acids and Precursors

| | | | Shorthand |
|---|---|---|---|
| Common Name | Abbreviation | Chemical Name | Notation |
| Myristic | — | tetradecanoic | 14:0 |
| Palmitic | Palmitate | hexadecanoic | 16:0 |
| Palmitoleic | — | 9-hexadecenoic | 16:1 |
| Stearic | — | octadecanoic | 18:0 |
| Oleic | — | cis-9-octadecenoic | 18:1 |
| Linoleic | LA | cis-9,12- | 18:2 omega-6 |
| | | octadecadienoic | |
| Gamma- | GLA | cis-6,9,12- | 18:3 omega-6 |
| Linolenic | | octadecatrienoic | |
| Eicosadienoic | EDA | cis-11,14- | 20:2 omega-6 |
| | | eicosadienoic | |
| Dihomo- | DGLA | cis-8,11,14- | 20:3 omega-6 |
| Gamma- | | eicosatrienoic | |
| Linolenic | | | |
| Arachidonic | ARA | cis-5,8,11,14- | 20:4 omega-6 |
| | | eicosatetraenoic | |
| Alpha-Linolenic | ALA | cis-9,12,15- | 18:3 omega-3 |
| | | octadecatrienoic | |
| Stearidonic | STA | cis-6,9,12,15- | 18:4 omega-3 |
| | | octadecatetraenoic | |
| Eicosatrienoic | ETrA | cis-11,14,17- | 20:3 omega-3 |
| | | eicosatrienoic | |
| Eicosa- | ETA | cis-8,11,14,17- | 20:4 omega-3 |
| tetraenoic | | eicosatetraenoic | |
| Eicosa- | EPA | cis-5,8,11,14,17- | 20:5 omega-3 |
| pentaenoic | | eicosapentaenoic | |
| Docosa- | DTA | cis-7,10,13,16- | 22:4 omega-3 |
| tetraenoic | | docosatetraenoic | |
| Docosa- | DPAn-6 | cis-4,7,10,13,16- | 22:5 omega-6 |
| pentaenoic | | docosapentaenoic | |
| Docosa- | DPAn-3 | cis-7,10,13,16,19- | 22:5 omega-3 |
| pentaenoic | | docosapentaenoic | |
| Docosa- | DHA | cis-4,7,10,13,16,19- | 22:6 omega-3 |
| hexaenoic | | docosahexaenoic | |

The term “high-level PUFA production” refers to production of at least about 25% PUFAs in the total lipids of the microbial host, preferably at least about 30% PUFAs in the total lipids, more preferably at least about 35% PUFA in the total lipids, more preferably at least about 40% PUFAs in the total lipids, more preferably at least about 40-45% PUFAs in the total lipids, more preferably at least about 45-50% PUFAs in the total lipids, more preferably at least about 50-60% PUFAs, and most preferably at least about 60-70% PUFAs in the total lipids. The structural form of the PUFA is not limiting; thus, for example, the PUFAs may exist in the total lipids as FFAs or in esterified forms such as acylglycerols, phospholipids, sulfolipids or glycolipids.

The term “oil-containing microbe” refers to a microorganism capable of producing a microbial oil. Thus, an oil-containing microbe may be yeast, algae, euglenoids, stramenopiles, fungi, or combinations thereof. In preferred embodiments, the oil-containing microbe is oleaginous.

The term “oleaginous” refers to those organisms that tend to store their energy source in the form of oil (Weete, In: Fungal Lipid Biochemistry, 2^{nd} Ed., Plenum, 1980). Generally, the cellular oil content of oleaginous microorganisms follows a sigmoid curve, wherein the concentration of lipid increases until it reaches a maximum at the late logarithmic or early stationary growth phase and then gradually decreases during the late stationary and death phases (Yongmanitchai and Ward, Appl. Environ. Microbiol., 57:419-25 (1991)). It is not uncommon for oleaginous microorganisms to accumulate in excess of about 25% of their dry cell weight as oil. Examples of oleaginous organisms include, but are not limited to organisms from a genus selected from the group consisting of Mortierella, Thraustochytrium, Schizochytrium, Yarrowia, Candida, Rhodotorula, Rhodosporidium, Cryptococcus, Trichosporon, and Lipomyces.

The term “oleaginous yeast” refers to those oleaginous microorganisms classified as yeasts that can make oil. Examples of oleaginous yeast include, but are no means limited to, the following genera: Yarrowia, Candida, Rhodotorula, Rhodosporidium, Cryptococcus, Trichosporon and Lipomyces.

In general, lipid accumulation in oleaginous microorganisms is triggered in response to the overall carbon to nitrogen ratio present in the growth medium. This process, leading to the de novo synthesis of free palmitate (16:0) in oleaginous microorganisms, is described in detail in U.S. Pat. No. 7,238,482. Palmitate is the precursor of longer-chain saturated and unsaturated fatty acid derivates, which are formed through the action of elongases and desaturases.

A wide spectrum of fatty acids (including saturated and unsaturated fatty acids and short-chain and long-chain fatty acids) can be incorporated into TAGs, the primary storage unit for fatty acids. Incorporation of long chain PUFAs into TAGs is most desirable, although the structural form of the PUFA is not limiting. More specifically, in one embodiment the oil-containing microbes will produce at least one PUFA selected from the group consisting of LA, GLA, EDA, DGLA, ARA, DTA, DPAn-6, ALA, STA, ETrA, ETA, EPA, DPAn-3, DHA and mixtures thereof. More preferably, the at least one PUFA has at least a C₂₀ chain length, such as PUFAs selected from the group consisting of EDA, DGLA, ARA, DTA, DPAn-6, ETrA, ETA, EPA, DPAn-3, DHA, and mixtures thereof. In one embodiment, the at least one PUFA is selected from the group consisting of ARA, EPA, DPAn-6, DPAn-3, DHA and mixtures thereof. In another preferred embodiment, the at least one PUFA is selected from the group consisting of EPA and DHA.

Most PUFAs are incorporated into TAGs as neutral lipids and are stored in lipid bodies. However, it is important to note that a measurement of the total PUFAs within an oleaginous organism should minimally include those PUFAs that are located in the phosphatidylcholine, phosphatidylethanolamine and TAG fractions.

Although the present invention is drawn to a process to form solid pellets comprising disrupted oil-containing microbes, which may optionally be subjected to extraction to produce microbial oil, one will appreciate an overview of the related processes that may be useful to obtain the oil-containing microbes themselves. Most processes will begin with a microbial fermentation, wherein a particular microorganism is cultured under conditions that permit growth and production of microbial oils. At an appropriate time, the microbial cells are harvested from the fermentation vessel. This untreated microbial biomass may be mechanically processed using various means, such as dewatering, drying, etc. Then, the process disclosed herein may then commence, wherein: (a) the microbial biomass is mixed with a grinding agent to provide a disrupted biomass mix; (b) a binding agent is blended with the disrupted biomass mix to provide a fixable mix; and, (c) the fixable mix is formed into a solid pellet. The solid pellets may optionally be subjected to oil extraction, producing residual biomass (e.g., cell debris in the form of a residual pellet) and extracted oil. Each of these aspects will be discussed in further detail below.

Oil-containing microbes produce microbial biomass as the microbes grow and multiply. The microbial biomass may be from any microorganism, whether naturally occurring or recombinant (“genetically engineered”), capable of producing a microbial oil. Thus, for example, oil-containing microbes may be selected from the group consisting of yeast, algae, euglenoids, stramenopiles, fungi, and mixtures thereof. Preferably, the microorganism will be capable of high level PUFA production within the microbial oil.

As an example, commercial sources of ARA oil are typically produced from microorganisms in the genera Mortierella (filamentous fungus), Entomophthora, Pythium and Porphyridium (red alga). Most notably, Martek Biosciences Corporation (Columbia, Md.) produces an ARA-containing fungal oil (ARASCO®; U.S. Pat. No. 5,658,767) which is substantially free of EPA and which is derived from either Mortierella alpina or Pythium insidiuosum.

Similarly, EPA can be produced microbially via numerous different processes based on the natural abilities of the specific microbial organism utilized [e.g., heterotrophic diatoms Cyclotella sp. and Nitzschia sp. (U.S. Pat. No. 5,244,921); Pseudomonas, Alteromonas or Shewanella species (U.S. Pat. No. 5,246,841); filamentous fungi of the genus Pythium (U.S. Pat. No. 5,246,842); Mortierella elongata, M. exigua, or M. hygrophila (U.S. Pat. No. 5,401,646); and eustigmatophycean alga of the genus Nannochloropsis (Krienitz, L. and M. Wirth, Limnologica, 36:204-210 (2006))].

DHA can also be produced using processes based on the natural abilities of native microbes. See, e.g., processes developed for Schizochytrium species (U.S. Pat. No. 5,340,742; U.S. Pat. No. 6,582,941); Ulkenia (U.S. Pat. No. 6,509,178); Pseudomonas sp. YS-180 (U.S. Pat. No. 6,207,441); Thraustochytrium genus strain LFF1 (U.S. 2004/0161831 A1); Crypthecodinium cohnii (U.S. Pat. Appl. Pub. No. 2004/0072330 A1; de Swaaf, M. E. et al., Biotechnol Bioeng., 81(6):666-72 (2003) and Appl. Microbiol. Biotechnol., 61(1):40-3 (2003)); Emiliania sp. (Japanese Patent Publication (Kokai) No. 5-308978 (1993)); and Japonochytrium sp. (ATCC #28207; Japanese Patent Publication (Kokai) No. 199588/1989)]. Additionally, the following microorganisms are known to have the ability to produce DHA: Vibrio marinus (a bacterium isolated from the deep sea; ATCC #15381); the micro-algae Cyclotella cryptica and Isochrysis galbana; and, flagellate fungi such as Thraustochytrium aureum (ATCC #34304; Kendrick, Lipids, 27:15 (1992)) and the Thraustochytrium sp. designated as ATCC #28211, ATCC #20890 and ATCC #20891. Currently, there are at least three different fermentation processes for commercial production of DHA: fermentation of C. cohnii for production of DHASCO™ (Martek Biosciences Corporation, Columbia, Md.); fermentation of Schizochytrium sp. for production of an oil formerly known as DHAGold (Martek Biosciences Corporation); and fermentation of Ulkenia sp. for production of DHActive™ (Nutrinova, Frankfurt, Germany).

Microbial production of PUFAs in microbial oils using recombinant means is expected to have several advantages over production from natural microbial sources. For example, recombinant microbes having preferred characteristics for oil production can be used, since the naturally occurring microbial fatty acid profile of the host can be altered by the introduction of new biosynthetic pathways in the host and/or by the suppression of undesired pathways, thereby resulting in increased levels of production of desired PUFAs (or conjugated forms thereof) and decreased production of undesired PUFAs. Secondly, recombinant microbes can provide PUFAs in particular forms which may have specific uses. Additionally, microbial oil production can be manipulated by controlling culture conditions, notably by providing particular substrate sources for microbially expressed enzymes, or by addition of compounds/genetic engineering to suppress undesired biochemical pathways. Thus, for example, it is possible to modify the ratio of omega-3 to omega-6 fatty acids so produced, or engineer production of a specific PUFA (e.g., EPA) without significant accumulation of other PUFA downstream or upstream products.

Thus, for example, a microbe lacking the natural ability to make EPA can be engineered to express a PUFA biosynthetic pathway by introduction of appropriate PUFA biosynthetic pathway genes, such as specific combinations of delta-4 desaturases, delta-5 desaturases, delta-6 desaturases, delta-12 desaturases, delta-15 desaturases, delta-17 desaturases, delta-9 desaturases, delta-8 desaturases, delta-9 elongases, C_{14/16} elongases, C_{16/18} elongases, C_{18/20} elongases and C_{20/22} elongases, although it is to be recognized that the specific enzymes (and genes encoding those enzymes) introduced are by no means limiting to the invention herein.

As an example, several yeast organisms have been recombinantly engineered to produce at least one PUFA. See for example, work in Saccharomyces cerevisiae (Dyer, J. M. et al., Appl. Eniv. Microbiol., 59:224-230 (2002); Domergue, F. et al., Eur. J. Biochem., 269:4105-4113 (2002); U.S. Pat. No. 6,136,574; U.S. Pat. Appl. Pub. No. 2006-0051847-A1) and the oleaginous yeast, Yarrowia lipolytica (U.S. Pat. No. 7,238,482; U.S. Pat. No. 7,465,564; U.S. Pat. No. 7,588,931; U.S. Pat. No. 7,932,077; U.S. Pat. No. 7,550,286; U.S. Pat. Appl. Pub. No. 2009-0093543-A1; and U.S. Pat. Appl. Pub. No. 2010-0317072-A1).

In some embodiments, advantages are perceived if the microbial host cells are oleaginous. Oleaginous yeast are naturally capable of oil synthesis and accumulation, wherein the total oil content can comprise greater than about 25% of the cellular dry weight, more preferably greater than about 30% of the cellular dry weight, and most preferably greater than about 40% of the cellular dry weight. In alternate embodiments, a non-oleaginous yeast can be genetically modified to become oleaginous such that it can produce more than 25% oil of the cellular dry weight, e.g., yeast such as Saccharomyces cerevisiae (Intl Appl. Pub. No. WO 2006/102342).

Genera typically identified as oleaginous yeast include, but are not limited to: Yarrowia, Candida, Rhodotorula, Rhodosporidium, Cryptococcus, Trichosporon and Lipomyces. More specifically, illustrative oil-synthesizing yeasts include: Rhodosporidium toruloides, Lipomyces starkeyii, L. lipoferus, Candida revkaufi, C. pulcherrima, C. tropicalis, C. utilis, Trichosporon pullans, T. cutaneum, Rhodotorula glutinus, R. graminis, and Yarrowia lipolytica (formerly classified as Candida lipolytica).

Most preferred is the oleaginous yeast Yarrowia lipolytica; and, in a further embodiment, most preferred are the Y. lipolytica strains designated as ATCC #20362, ATCC #8862, ATCC #18944, ATCC #76982 and/or LGAM S(7)1 (Papanikolaou S., and Aggelis G., Bioresour. Technol. 82(1):43-9 (2002)).

In some embodiments, it may be desirable for the oleaginous yeast to be capable of “high-level PUFA production”, wherein the organism can produce at least about 5-10% of the desired PUFA (i.e., LA, ALA, EDA, GLA, STA, ETrA, DGLA, ETA, ARA, DPA n-6, EPA, DPA n-3 and/or DHA) in the total lipids. More preferably, the oleaginous yeast will produce at least about 10-70% of the desired PUFA(s) in the total lipids. Although the structural form of the PUFA is not limiting, preferably TAGs comprise the PUFA(s).

Thus, the PUFA biosynthetic pathway genes and gene products described herein may be produced in heterologous microbial host cells, particularly in the cells of oleaginous yeasts (e.g., Yarrowia lipolytica). Expression in recombinant microbial hosts may be useful for the production of various PUFA pathway intermediates, or for the modulation of PUFA pathways already existing in the host for the synthesis of new products heretofore not possible using the host.

Although numerous oleaginous yeast could be engineered for production of preferred omega-3/omega-6 PUFAs based on the cited teachings provided above, representative PUFA-producing strains of the oleaginous yeast Yarrowia lipolytica are described in Table 3. These strains possess various combinations of the following PUFA biosynthetic pathway genes: delta-4 desaturases, delta-5 desaturases, delta-6 desaturases, delta-12 desaturases, delta-15 desaturases, delta-17 desaturases, delta-9 desaturases, delta-8 desaturases, delta-9 elongases, C_{14/16} elongases, C_{16/18} elongases, C_{18/20} elongases and C_{20/22} elongases, although it is to be recognized that the specific enzymes (and genes encoding those enzymes) introduced and the specific PUFAs produced are by no means limiting to the invention herein.

TABLE 3
Lipid Profiles of Representative Yarrowia lipolytica Strains Engineered to Produce Omega-3/Omega-6 PUFAs

| | | ATCC | Fatty Acid Content (As A Percent [%] of Total Fatty Acids) | | | | | | | | | | | | | | TFAs |
|---|---|---|---|---|---|---|---|---|---|---|---|---|---|---|---|---|---|
| | | Deposit | | | | | | 18:3 | | 20:2 | | | | | DPA | | % |
| Strain | Reference | No. | 16:0 | 16:1 | 18:0 | 18:1 | 18:2 | (ALA) | GLA | (EDA) | DGLA | ARA | ETA | EPA | n-3 | DHA | DCW |
| Wildtype | U.S. Pat. | #76982 | 14 | 11 | 3.5 | 34.8 | 31 | 0 | 0 | — | — | — | — | — | — | — | — |
| pDMW208 | No. | — | 11.9 | 8.6 | 1.5 | 24.4 | 17.8 | 0 | 25.9 | — | — | — | — | — | — | — | — |
| pDMW208- | 7,465,564 | — | 16.2 | 1.5 | 0.1 | 17.8 | 22.2 | 0 | 34 | — | — | — | — | — | — | — | — |
| D62 | | | | | | | | | | | | | | | | | |
| M4 | U.S. Pat. | — | 15 | 4 | 2 | 5 | 27 | 0 | 35 | — | 8 | 0 | 0 | 0 | — | — | — |
| | No. | | | | | | | | | | | | | | | | |
| | 7,932,077 | | | | | | | | | | | | | | | | |
| Y2034 | U.S. Pat. | — | 13.1 | 8.1 | 1.7 | 7.4 | 14.8 | 0 | 25.2 | — | 8.3 | 11.2 | — | — | — | — | — |
| Y2047 | No. | PTA- | 15.9 | 6.6 | 0.7 | 8.9 | 16.6 | 0 | 29.7 | — | 0 | 10.9 | — | — | — | — | — |
| | 7,588,931 | 7186 | | | | | | | | | | | | | | | |
| Y2214 | | — | 7.9 | 15.3 | 0 | 13.7 | 37.5 | 0 | 0 | — | 7.9 | 14 | — | — | — | — | — |
| EU | U.S. Pat. | — | 19 | 10.3 | 2.3 | 15.8 | 12 | 0 | 18.7 | — | 5.7 | 0.2 | 3 | 10.3 | — | — | 36 |
| Y2072 | No. | — | 7.6 | 4.1 | 2.2 | 16.8 | 13.9 | 0 | 27.8 | — | 3.7 | 1.7 | 2.2 | 15 | — | — | — |
| Y2102 | 7,932,077 | — | 9 | 3 | 3.5 | 5.6 | 18.6 | 0 | 29.6 | — | 3.8 | 2.8 | 2.3 | 18.4 | — | — | — |
| Y2088 | | — | 17 | 4.5 | 3 | 2.5 | 10 | 0 | 20 | — | 3 | 2.8 | 1.7 | 20 | — | — | — |
| Y2089 | | — | 7.9 | 3.4 | 2.5 | 9.9 | 14.3 | 0 | 37.5 | — | 2.5 | 1.8 | 1.6 | 17.6 | — | — | — |
| Y2095 | | — | 13 | 0 | 2.6 | 5.1 | 16 | 0 | 29.1 | — | 3.1 | 1.9 | 2.7 | 19.3 | — | — | — |
| Y2090 | | — | 6 | 1 | 6.1 | 7.7 | 12.6 | 0 | 26.4 | — | 6.7 | 2.4 | 3.6 | 26.6 | — | — | 22.9 |
| Y2096 | | PTA- | 8.1 | 1 | 6.3 | 8.5 | 11.5 | 0 | 25 | — | 5.8 | 2.1 | 2.5 | 28.1 | — | — | 20.8 |
| | | 7184 | | | | | | | | | | | | | | | |
| Y2201 | | PTA- | 11 | 16.1 | 0.7 | 18.4 | 27 | 0 | — | 3.3 | 3.3 | 1 | 3.8 | 9 | — | — | — |
| | | 7185 | | | | | | | | | | | | | | | |
| Y3000 | U.S. Pat. | PTA- | 5.9 | 1.2 | 5.5 | 7.7 | 11.7 | 0 | 30.1 | — | 2.6 | 1.2 | 1.2 | 4.7 | 18.3 | 5.6 | — |
| | No. | 7187 | | | | | | | | | | | | | | | |
| | 7,550,286 | | | | | | | | | | | | | | | | |
| Y4001 | U.S. Pat. | — | 4.3 | 4.4 | 3.9 | 35.9 | 23 | 0 | — | 23.8 | 0 | 0 | 0 | — | — | — | — |
| Y4036 | Appl. Pub. | — | 7.7 | 3.6 | 1.1 | 14.2 | 32.6 | 0 | — | 15.6 | 18.2 | 0 | 0 | — | — | — | — |
| Y4070 | No. 2009- | — | 8 | 5.3 | 3.5 | 14.6 | 42.1 | 0 | — | 6.7 | 2.4 | 11.9 | — | — | — | — | — |
| Y4086 | 0093543- | — | 3.3 | 2.2 | 4.6 | 26.3 | 27.9 | 6.9 | — | 7.6 | 1 | 0 | 2 | 9.8 | — | — | 28.6 |
| Y4128 | A1 | PTA- | 6.6 | 4 | 2 | 8.8 | 19 | 2.1 | — | 4.1 | 3.2 | 0 | 5.7 | 42.1 | — | — | 18.3 |
| | | 8614 | | | | | | | | | | | | | | | |
| Y4158 | | — | 3.2 | 1.2 | 2.7 | 14.5 | 30.4 | 5.3 | — | 6.2 | 3.1 | 0.3 | 3.4 | 20.5 | — | — | 27.3 |
| Y4184 | | — | 3.1 | 1.5 | 1.8 | 8.7 | 31.5 | 4.9 | — | 5.6 | 2.9 | 0.6 | 2.4 | 28.9 | — | — | 23.9 |
| Y4217 | | — | 3.9 | 3.4 | 1.2 | 6.2 | 19 | 2.7 | — | 2.5 | 1.2 | 0.2 | 2.8 | 48.3 | — | — | 20.6 |
| Y4259 | | — | 4.4 | 1.4 | 1.5 | 3.9 | 19.7 | 2.1 | — | 3.5 | 1.9 | 0.6 | 1.8 | 46.1 | — | — | 23.7 |
| Y4305 | | — | 2.8 | 0.7 | 1.3 | 4.9 | 17.6 | 2.3 | — | 3.4 | 2 | 0.6 | 1.7 | 53.2 | — | — | 27.5 |
| Y4127 | Int'l. App. | PTA- | 4.1 | 2.3 | 2.9 | 15.4 | 30.7 | 8.8 | — | 4.5 | 3.0 | 3.0 | 2.8 | 18.1 | — | — | — |
| | Pub. No. | 8802 | | | | | | | | | | | | | | | |
| | WO | | | | | | | | | | | | | | | | |
| Y4184 | 2008/ | — | 2.2 | 1.1 | 2.6 | 11.6 | 29.8 | 6.6 | — | 6.4 | 2.0 | 0.4 | 1.9 | 28.5 | — | — | 24.8 |
| | 073367 | | | | | | | | | | | | | | | | |
| Y8404 | U.S. Pat. | — | 2.8 | 0.8 | 1.8 | 5.1 | 20.4 | 2.1 | | 2.9 | 2.5 | 0.6 | 2.4 | 51.1 | — | — | 27.3 |
| Y8406 | Appl. Pub. | PTA- | 2.6 | 0.5 | 2.9 | 5.7 | 20.3 | 2.8 | | 2.8 | 2.1 | 0.5 | 2.1 | 51.2 | — | — | 30.7 |
| | No. 2010- | 10025 | | | | | | | | | | | | | | | |
| Y8412 | 0317072- | PTA- | 2.5 | 0.4 | 2.6 | 4.3 | 19.0 | 2.4 | | 2.2 | 2.0 | 0.5 | 1.9 | 55.8 | — | — | 27.0 |
| | A1 | 10026 | | | | | | | | | | | | | | | |
| Y8647 | | — | 1.3 | 0.2 | 2.1 | 4.7 | 20.3 | 1.7 | | 3.3 | 3.6 | 0.7 | 3.0 | 53.6 | — | — | 37.6 |
| Y9028 | | — | 1.3 | 0.2 | 2.1 | 4.4 | 19.8 | 1.7 | | 3.2 | 2.5 | 0.8 | 1.9 | 54.5 | — | — | 39.6 |
| Y9477 | | — | 2.6 | 0.5 | 3.4 | 4.8 | 10.0 | 0.5 | | 2.5 | 3.7 | 1.0 | 2.1 | 61.4 | — | — | 32.6 |
| Y9497 | | — | 2.4 | 0.5 | 3.2 | 4.6 | 11.3 | 0.8 | | 3.1 | 3.6 | 0.9 | 2.3 | 58.7 | — | — | 33.7 |
| Y9502 | | — | 2.5 | 0.5 | 2.9 | 5.0 | 12.7 | 0.9 | | 3.5 | 3.3 | 0.8 | 2.4 | 57.0 | — | — | 37.1 |
| Y9508 | | — | 2.3 | 0.5 | 2.7 | 4.4 | 13.1 | 0.9 | | 2.9 | 3.3 | 0.9 | 2.3 | 58.7 | — | — | 34.9 |
| Y8145 | | — | 4.3 | 1.7 | 1.4 | 4.8 | 18.6 | 2.8 | | 2.2 | 1.5 | 0.6 | 1.5 | 48.5 | — | — | 23.1 |
| Y8259 | | PTA- | 3.5 | 1.3 | 1.3 | 4.8 | 16.9 | 2.3 | | 1.9 | 1.7 | 0.6 | 1.6 | 53.9 | — | — | 20.5 |
| | | 10027 | | | | | | | | | | | | | | | |
| Y8370 | | — | 3.4 | 1.1 | 1.4 | 4.0 | 15.7 | 1.9 | | 1.7 | 1.9 | 0.6 | 1.5 | 56.4 | — | — | 23.3 |
| Y8672 | | — | 2.3 | 0.4 | 2.0 | 4.0 | 16.1 | 1.4 | | 1.8 | 1.6 | 0.7 | 1.1 | 61.8 | — | — | 26.5 |

One of skill in the art will appreciate that the methodology of the present invention is not limited to the Yarrowia lipolytica strains described above, nor to the species (i.e., Yarrowia lipolytica) or genus (i.e., Yarrowia) in which the invention has been demonstrated, as the means to introduce a PUFA biosynthetic pathway into an oleaginous yeast are well known. Instead, any oleaginous yeast or any other suitable microbe capable of producing PUFAs will be equally suitable for use in the present methodologies, as demonstrated in Example 11 (although some process optimization may be required for each new microbe handled, based on differences in, e.g., the cell wall composition of each microbe).

A microbial species producing a lipid, preferably comprising a PUFA(s), may be cultured and grown in a fermentation medium under conditions whereby the lipid is produced by the microorganism. Typically, the microorganism is fed with a carbon and nitrogen source, along with a number of additional chemicals or substances that allow growth of the microorganism and/or production of the microbial oil (preferably comprising PUFAs). The fermentation conditions will depend on the microorganism used, as described in the above citations, and may be optimized for a high content of the PUFA(s) in the resulting biomass.

In general, media conditions may be optimized by modifying the type and amount of carbon source, the type and amount of nitrogen source, the carbon-to-nitrogen ratio, the amount of different mineral ions, the oxygen level, growth temperature, pH, length of the biomass production phase, length of the oil accumulation phase and the time and method of cell harvest. For example, Yarrowia lipolytica are generally grown in a complex media such as yeast extract-peptone-dextrose broth (YPD) or a defined minimal media (e.g., Yeast Nitrogen Base (DIFCO Laboratories, Detroit, Mich.) that lacks a component necessary for growth and thereby forces selection of the desired recombinant expression cassettes that enable PUFA production).

When the desired amount of microbial oil, preferably comprising PUFAs, has been produced by the microorganism, the fermentation medium may be mechanically processed to obtain untreated microbial biomass comprising the microbial oil. For example, the fermentation medium may be filtered or otherwise treated to remove at least part of the aqueous component. As will be appreciated by those in the art, the untreated microbial biomass typically includes water. Preferably, a portion of the water is removed from the untreated microbial biomass after microbial fermentation to provide a microbial biomass with a moisture level of less than 10 weight percent, more preferably a moisture level of less than 5 weight percent, and most preferably a moisture level of 3 weight percent or less. The microbial biomass moisture level can be controlled in drying. Preferably the microbial biomass has a moisture level in the range of about 1 to 10 weight percent.

Optionally, the fermentation medium and/or the microbial biomass may be pasteurized or treated via other means to reduce the activity of endogenous microbial enzymes that can harm the microbial oil and/or PUFA products.

Thus, the microbial biomass may be in the form of whole cells, whole cell lysates, homogenized cells, partially hydrolyzed cellular material, and/or disrupted cells (i.e., disrupted microbial biomass).

The disrupted microbial biomass will have a disruption efficiency of at least 50% of the oil-containing microbes. More preferably, the disruption efficiency is at least 70%, more preferably at least 80% and most preferably 85-90% or more, of the oil-containing microbes. Although preferred ranges are described above, useful examples of disruption efficiencies include any integer percentage from 50% to 100%, such as 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% disruption efficiency.

The disruption efficiency refers to the percent of cells walls that have been fractured or ruptured during processing, as determined qualitatively by optical visualization or as determined quantitatively according to the following formula: disruption efficiency=(% free oil*100) divided by (% total oil), wherein % free oil and % total oil are measured for the solid pellet.

A solid pellet that has been not subjected to a process of disruption (e.g., mechanical crushing using e.g., screw extrusion, an expeller, pistons, bead beaters, mortar and pestle, Hammer-milling, air-jet milling, etc.) will typically have a low disruption efficiency since fatty acids within DAGs, MAGs and TAGs, phosphatidylcholine and phosphatidylethanolamine fractions and free fatty acids, etc. are generally not extractable from the microbial biomass until a process of disruption has broken both cell walls and internal membranes of various organelles, including membranes surrounding lipid bodies. Various processes of disruption will result in various disruption efficiencies, based on the particular shear, compression, static and dynamic forces inherently produced in the process

Increased disruption efficiency of the microbial biomass typically leads to increased extraction yields (e.g., as measured by the weight percent of crude extracted oil), likely since more of the microbial oil is susceptible to the presence of the extraction solvent(s) with disruption of cell walls and membranes.

Although a variety of equipment may be utilized to produce the disrupted microbial biomass, preferably the disrupting is performed in a twin screw extruder. More specifically, the twin screw extruder preferably comprises: (i) a total specific energy input (SEI) in the extruder of about 0.04 to 0.4 KW/(kg/hr), more preferably 0.05 to 0.2 KW/(kg/hr) and most preferably about 0.07 to 0.15 KW/(kg/hr); (ii) a compaction zone using bushing elements with progressively shorter pitch length; and, (iii) a compression zone using flow restriction. Most of the mechanical energy required for cell disruption is imparted in the compression zone, which is created using flow restriction in the form of e.g., reverse screw elements, restriction/blister ring elements or kneading elements. The compaction zone is prior to the compression zone within the extruder. A first zone of the extruder may be present to feed and transport the biomass into the compaction zone.

Step (a) of the present invention comprises a step of mixing a microbial biomass, having a moisture level and comprising oil-containing microbes, and at least one grinding agent capable of absorbing oil, to provide a disrupted biomass mix.

The grinding agent, capable of absorbing oil, may be a particle having a Moh hardness of 2.0 to 6.0, and preferably 2.0 to about 5.0; and more preferably about 2.0 to 4.0; and an oil absorption coefficient of 0.8 or higher, preferably 1.0 or higher, and more preferably 1.3 or higher, as determined according to the American Society for Testing And Materials (ASTM) Method D1483-60. Preferred grinding agents have a median particle diameter of about 2 to 20 microns, and preferably about 7 to 10 microns; and a specific surface area of at least 1 m²/g and preferably 2 to 100 m²/g as determined with the BET method (Brunauer, S. et al. J. Am. Chem. Soc., 60:309 (1938)).

Preferred grinding agents are selected from the group consisting of silica and silicate. As used herein, the term “silica” refers to a solid chemical substance consisting mostly (at least 90% and preferably at least 95% by weight) of silicon and oxygen atoms in a ratio of about two oxygen atoms to one silicon atom, thus having the empirical formula of SiO₂. Silicas include, for example, precipitated silicas, fumed silicas, amorphous silicas, diatomaceous silicas, also known as diatomaceous earths (D-earth) as well as silanized forms of these silicas. The term “silicate” refers to a solid chemical substance consisting mostly (at least 90% and preferably at least 95% by weight) of atoms of silicon, oxygen and at least one metal ion. The metal ion may be, for instance, lithium, sodium, potassium, magnesium, calcium, aluminum, or a mixture thereof. Aluminum silicates in the form of zeolites, natural and synthetic, may be used. Other silicates that may be useful are calcium silicates, magnesium silicates, sodium silicates, and potassium silicates.

A preferred grinding agent is diatomaceous earth (D-earth) having a specific surface area of about 10-20 m²/g and an oil absorption coefficient of 1.3 or higher. A commercial source of a suitable grinding agent capable of absorbing oil is Celite 209 D-earth available from Celite Corporation, Lompoc, Calif.

Other grinding agents may be poly(meth)acrylic acids, and ionomers derived from partial or full neutralization of poly(meth)acrylic acids with sodium or potassium bases. Herein the term (meth)acrylate means the compound may be either an acrylate, a methacrylate, or a mixture of the two.

The at least one grinding agent is present at about 1 to 20 weight percent, more preferably 1 to 15 weight percent, and most preferably about 2 to 12 weight percent, based on the summation of components (a) microbial biomass, (b) grinding agent and (c) binding agent in the solid pellet.

Mixing a microbial biomass and a grinding agent capable of absorbing oil to provide a disrupted biomass mix [step (a)] can be performed by any method known in the art to apply energy to a mixing media. Preferably the mixing provides a disrupted biomass mix having a temperature of 90° C. or less, and more preferably 70° C. or less.

For example, the microbial biomass and grinding agent may be fed into a mixer, such as a single screw extruder or twin screw extruder, agitator, single screw or twin screw kneader, or Banbury mixer, and the addition step may be addition of all ingredients at once or gradual addition in batches.

Preferably the mixing is performed in a twin screw extruder, as described above, having a SEI of about 0.04 to 0.4 KW/(kg/hr), a compaction zone using bushing elements with progressively shorter pitch length, and a compression zone using flow restriction. Under these conditions, the initial microbial biomass may be whole dried cells and the process of cell disruption, resulting in a disrupted microbial biomass having a disruption efficiency of at least 50% of the oil-containing microbes, may occur at the beginning or during the mixing step, that is, cell disruption and step (a) may be combined and simultaneous to produce a disrupted biomass mix. The presence of the grinding agent enhances cell disruption; however, most cell disruption occurs as a result of the twin screw extruder itself.

Thus, for clarity, cell disruption of the microbial biomass can be performed in the absence of grinding agent, for instance in a twin screw extruder having a compression zone as disclosed above and then mixing of grinding agent and disrupted microbial biomass can be performed in the twin screw extruder or a variety of other mixers to provide the disrupted biomass mix. Or, cell disruption of the microbial biomass can be performed in the presence of grinding agent, for instance in a twin screw extruder having a compression zone. In either case, however, cell disruption (i.e., disruption efficiency) should be maximized if one desires to maximize the yield of extracted oil from the oil-containing microbes in subsequent process steps.

Step (b) of the present invention comprises a step of blending a binding agent with said disrupted biomass mix to provide a fixable mix capable of forming a solid pellet.

Binding agents useful in the invention include hydrophilic organic materials and hydrophilic inorganic materials that are water soluble or water dispersible. Preferred water soluble binding agents have solubility in water of at least 1 weight percent, preferably at least 2 weight percent and more preferably at least 5 weight percent, at 23° C.

The binding agent preferably has solubility in supercritical fluid carbon dioxide at 500 bar of less than 1×10⁻³ mol fraction; and preferably less than 1×10⁻⁴, more preferably less than 1×10⁻⁵, and most preferably less than 1×10⁻⁶ mol fraction. The solubility may be determined according to the methods disclosed in “Solubility in Supercritical Carbon Dioxide”, Ram Gupta and Jae-Jin Shim, Eds., CRC (2007).

The binding agent acts to retain the integrity and size of pellets formed from the pelletization process and furthermore acts to reduce fines in further processing and transport of the pellets.

Suitable organic binding agents include: alkali metal carboxymethyl cellulose with degrees of substitution of 0.5 to 1; polyethylene glycol and/or alkyl polyethoxylate, preferably with an average molecular weight below 1,000; phosphated starches; cellulose and starch ethers, such as carboxymethyl starch, methyl cellulose, hydroxyethyl cellulose, hydroxypropyl cellulose and corresponding cellulose mixed ethers; proteins including gelatin and casein; polysaccharides including tragacanth, sodium and potassium alginate, guam Arabic, tapioca, partly hydrolyzed starch including maltodextrose and dextrin, and soluble starch; sugars including sucrose, invert sugar, glucose syrup and molasses; synthetic water-soluble polymers including poly(meth)acrylates, copolymers of acrylic acid with maleic acid or compounds containing vinyl groups, polyvinyl alcohol, partially hydrolyzed polyvinyl acetate and polyvinyl pyrrolidone. If the compounds mentioned above are those containing free carboxyl groups, they are normally present in the form of their alkali metal salts, more particularly their sodium salts.

Phosphated starch is understood to be a starch derivative in which hydroxyl groups of the starch anhydroglucose units are replaced by the group —O—P(O)(OH)₂ or water-soluble salts thereof, more particularly alkali metal salts, such as sodium and/or potassium salts. The average degree of phosphation of the starch is understood to be the number of esterified oxygen atoms bearing a phosphate group per saccharide monomer of the starch averaged over all the saccharide units. The average degree of phosphation of preferred phosphate starches is in the range from 1.5 to 2.5.

Partly hydrolyzed starches in the context of the present invention are understood to be oligomers or polymers of carbohydrates which may be obtained by partial hydrolysis of starch using conventional, for example acid- or enzyme-catalyzed processes. The partly hydrolyzed starches are preferably hydrolysis products with average molecular weights of 440 to 500,000. Polysaccharides with a dextrose equivalent (DE) of 0.5 to 40 and, more particularly, 2 to 30 are preferred, DE being a standard measure of the reducing effect of a polysaccharide by comparison with dextrose (which has a DE of 100, i.e., DE 100). Both maltodextrins (DE 3-20) and dry glucose syrups (DE 20-37) and also so-called yellow dextrins and white dextrins with relatively high average molecular weights of about 2,000 to 30,000 may be used after phosphation.

A preferred class of binding agent is water and carbohydrates selected from the group consisting of sucrose, lactose, fructose, glucose, and soluble starch. Preferred binding agents have a melting point of at least 50° C., preferably at least 80° C., and more preferably at least 100° C.

Suitable inorganic binding agents include sodium silicate, bentonite, and magnesium oxide.

Preferred binding agents are materials that are considered “food grade” or “generally recognized as safe” (GRAS).

The binding agent is present at about 0.5 to 10 weight percent, preferably 1 to 10 weight percent, and more preferably about 3 to 8 weight percent, based on the summation of components (a) microbial biomass, (b) grinding agent and (c) binding agent in the solid pellet.

As one of skill in the art will appreciate, fixable mix (i.e., obtained by blending the disrupted biomass mix with at least one binding agent) will have significantly higher moisture level than the moisture level of the final solid pellet, to permit ease of handling (e.g., extruding the fixable mix into a die). Thus, for example, a binding agent comprising a solution of sucrose and water can be added to the disrupted biomass mix in a manner that results in a fixable mix having within 0.5 to 20 weight percent water. However, upon drying of the fixable mix to form a solid pellet, the final moisture level of the solid pellet is less than 5 weight percent of water and the sucrose is less than 10 weight percent.

Blending the at least one binding agent with disrupted biomass mix to provide a fixable mix [step (b)] can be performed by any method that allows dissolution of the binding agent and blending with the disrupted biomass to provide a fixable mix. The term “fixable mix” means that the mix is capable of forming a solid pellet upon removal of solvent, for instance water, in a drying step.

The binding agent can be blended by a variety of means. One method includes dissolution of the binding agent in a solvent to provide a binder solution, following by metering the binder solution, at a controlled rate, into the disrupted biomass mix. A preferred solvent is water, but other solvents, for instance ethanol, isopropanol, and such, may be used advantageously. Another method includes adding the binding agent, as a solid or solution, to the biomass/grinding agent at the beginning or during the mixing step, that is, step (a) and (b) are combined and simultaneous. If the binding agent is added as a solid, preferably sufficient moisture is present in the disrupted biomass mix to dissolve the binding agent during the blending step. A preferred method of blending includes metering the binder solution, at a controlled rate, into the disrupted biomass mix in an extruder, preferably after the compression zone, as disclosed above. The addition of a binder solution after the compression zone allows for rapid cooling of the disrupted biomass mix.

Forming solid pellets from the fixable mix [step (c)] can be performed by a variety of means known in the art. One method includes extruding the fixable mix into a die, for instance a dome granulator, to form strands of uniform diameter that are dried on a vibrating or fluidized bed drier to break the strands to provide pellets. The pelletized material is suitable for downstream oil extraction, transport, or other purposes.

The solid pellets provided by the process disclosed herein desirably are non-tacky at room temperature. A large plurality of the solid pellets may be packed together for many days without degradation of the pellet structure, and without binding together. A large plurality of pellets desirably is a free-flowing pelletized composition. Preferably the pellets have an average diameter of about 0.5 to about 1.5 mm and an average length of about 2.0 to about 8.0 mm. Preferably, the solid pellets have a final moisture level of about 0.1% to 5.0%, with a range about 0.5% to 3.0% more preferred. Increased moisture levels in the final solid pellets may lead to difficulties during storage due to growth of e.g., molds.

In one embodiment, the present invention is thus drawn to a pelletized oil-containing microbial biomass made by the process of steps (a)-(c), as disclosed above.

Also disclosed is a solid pellet comprising:
a) about 70 to about 98.5 weight percent of disrupted biomass comprising oil-containing microbes;
b) about 1 to about 20 weight percent grinding agent capable of absorbing oil; and,
c) about 0.5 to 10 weight percent binding agent;
   wherein the weight percents are based on the summation of (a), (b) and (c) in the solid pellet. The solid pellet may comprise 75 to 98 weight percent (a); 1 to 15 weight percent (b) and 1 to 10 weight percent (c); and, preferably the pellet comprises 80 to 95 weight percent (a); 2 to 12 weight percent (b) and 3 to 8 weight percent (c).

Another embodiment of the invention herein is the process of steps (a)-(c) as disclosed above, further comprising step (d), i.e., extracting the solid pellet with a solvent to provide an extracted oil and an extracted pellet (i.e., “residual biomass” or “residual pellet”).

Oil extraction can occur via treatment with various organic solvents (e.g., hexane, iso-hexane), enzymatic extraction, osmotic shock, ultrasonic extraction, supercritical fluid extraction (e.g., CO₂ extraction), saponification and combinations of these methods.

In one preferred embodiment, extraction occurs using supercritical fluids (SCFs). SCFs exhibit properties intermediate between those of gases and liquids. A key feature of a SCF is that the fluid density can be varied continuously from liquid-like to gas-like densities by varying either the temperature or pressure, or a combination thereof. Various density-dependent physical properties likewise exhibit similar continuous variation in this region. Some of these properties include, but are not limited to, solvent strength (as evidenced by the solubilities of various substances in the SCF media), polarity, viscosity, diffusivity, heat capacity, thermal conductivity, isothermal compressibility, expandability, contractibility, fluidity, and molecular packing. The density variation in a SCF also influences the chemical potential of solutes and hence, reaction rates and equilibrium constants. Thus, the solvent environment in a SCF media can be optimized for a specific application by tuning the various density-dependent fluid properties.

A fluid is in the SCF state when the system temperature and pressure exceed the corresponding critical point values defined by the critical temperature (Tₐ) and pressure (P_{c}). For pure substances, the critical temperature and pressure are the highest at which vapor and liquid phases can coexist. Above the critical temperature, a liquid does not form for a pure substance, regardless of the applied pressure. Similarly, the critical pressure and critical molar volume are defined at this critical temperature corresponding to the state at which the vapor and liquid phases merge. Although more complex for multicomponent mixtures, a mixture critical state is similarly identified as the condition at which the properties of coexisting vapor and liquid phases become indistinguishable. For a discussion of supercritical fluids, see Kirk-Othmer Encycl. of Chem. Technology, 4^{th} ed., Vol. 23, pg. 452-477, John Wiley & Sons, NY (1997).

Any suitable SCF or liquid solvent may be used in the oil extraction step, e.g., the contacting of the solid pellets with a solvent to separate the oil from the microbial biomass, including, but not limited to, CO₂, tetrafluoromethane, ethane, ethylene, propane, propylene, butane, isobutane, isobutene, pentane, hexane, cyclohexane, benzene, toluene, xylenes, and mixtures thereof, provided that it is inert to all reagents and products. Preferred solvents include CO₂ or a C₃-C₆ alkane. More preferred solvents are CO₂, pentane, butane, and propane. Most preferred solvents are supercritical fluid solvents comprising CO₂.

In a preferred embodiment, super-critical CO₂ extraction is performed, as disclosed in U.S. Pat. Pub. No. 2011-0263709-A1, entitled “Method for Obtaining Polyunsaturated Fatty Acid-Containing Compositions from Biomass” (hereby incorporated herein by reference). This particular methodology subjects the microbial biomass to oil extraction to remove phospholipids (PLs) and residual biomass, and then fractionates the resulting extract to produce an extracted oil having a “refined lipid composition”. The refined lipid composition may comprise neutral lipids and/or free fatty acids while being substantially free of PLs. The refined lipid composition may be enriched in TAGs (comprising PUFAs) relative to the oil composition of the microbial biomass. The refined lipid composition may undergo further purification to produce a “purified oil”.

Thus, the extracted oil comprises a lipid fraction substantially free of PLs, and the extracted residual pellet comprising residual biomass comprises PLs. In this method, the supercritical fluids comprising CO₂ may further comprise at least one additional solvent (i.e., a cosolvent), for example one or more of the solvents listed above, as long as the presence or amount of the additional solvent is not deleterious to the process, for example does not solubilize the PLs contained in the microbial biomass during the primary extraction step. However, a polar cosolvent such as ethanol, methanol, acetone, or the like may be added to intentionally impart polarity to the solvent phase to enable extraction of the PLs from the microbial biomass during optional secondary oil extractions to isolate the PLs.

The solid pellets comprising oil-containing disrupted microbial biomass may be contacted with liquid or supercritical CO₂ under suitable extraction conditions to provide an extract and a residual biomass according to at least two methods. According to a first method of U.S. Pat. Pub. No. 2011-0263709-A1, contacting the untreated microbial biomass with CO₂ is performed multiple times under extraction conditions corresponding to increasing solvent density, for example under increasing pressure and/or decreasing temperature, to obtain extracts comprising a refined lipid composition wherein the lipid fractions are substantially free of PLs. The refined lipid composition of the extracts varies in the distribution of FFAs, MAGs, DAGs, and TAGs according to their relative solubilities, which depend upon the solvent density corresponding to the selected extraction conditions of each of the multiple extractions.

Alternatively and according to the present methods, in a second method of U.S. Pat. Pub. No. 2011-0263709-A1, the untreated microbial biomass is contacted with a solvent such as CO₂ under extraction conditions selected to provide an extract comprising a lipid fraction substantially free of PLs, which subsequently undergoes a series of multiple staged pressure letdown steps to provide refined lipid compositions. Each of these staged pressure letdown steps is conducted in a separator vessel at pressure and temperature conditions corresponding to decreasing solvent density to isolate a liquid-phase refined lipid composition which can be separated from the extract phase by, for example, simple decantation. The refined lipid compositions which are provided vary in the distribution of FFAs, MAGs, DAGs, and TAGs according to their relative solubilities, which depend upon the solvent density corresponding to the selected conditions of the staged separator vessels.

The refined lipid compositions obtained by the second method may correspond to the extracts obtained in the first method when extraction conditions are appropriately matched. It is thus believed possible to exemplify the refined lipid compositions obtainable by the present method through performance of the first method.

According to the present methods, the solid pellets comprising oil-containing disrupted microbial biomass may be contacted with a solvent such as liquid or SCF CO₂ at a temperature and pressure and for a contacting time sufficient to obtain an extract comprising a lipid fraction substantially free of PLs. The lipid fraction may comprise neutral lipids (e.g., comprising TAGs, DAGs, and MAGs) and FFAs. The contacting and fractionating temperatures may be chosen to provide liquid or SCF CO₂, to be within the thermal stability range of the PUFA(s), and to provide sufficient density of the CO₂ to solubilize the TAGs, DAGs, MAGs, and FFAs. Generally, the contacting and fractionating temperatures may be from about 20° C. to about 100° C., for example from about 35° C. to about 100° C.; the pressure may be from about 60 bar to about 800 bar, for example from about 80 bar to about 600 bar. A sufficient contacting time, as well as appropriate CO₂ to biomass ratios, may be determined by generating extraction curves for a particular sample of solid pellets. These extraction curves are dependent upon the extraction conditions of temperature, pressure, CO₂ flow rate, and variables such as the extent of cell disruption and the form of the biomass. In one embodiment of the present methods, the solvent comprises liquid or supercritical fluid CO₂ and the mass ratio of CO₂ to the microbial biomass is from about 20:1 to about 70:1, for example from about 20:1 to about 50:1.

The methodology of the present invention has proven to be effective, highly scale-able, robust and user-friendly, while allowing production at relatively high yields and at high throughput rates. Cell disruption using conventional techniques such as spray drying, use of high shear mixers, etc. was found to be inadequate for e.g., yeast cell walls comprising chitin. Incumbent wet media mill disruption process produced fines and colloidal contamination which necessitated further separation steps and resulted in significant oil loss. Additionally, wet media milling steps introduced a liquid carrier (e.g., isohexane or water) which complicated downstream processing by requiring liquid-solid separation step with oil losses. The process described herein relies on the production of a disrupted biomass mix (i.e., wherein the disrupted biomass mix is produced by mixing a microbial biomass, having a moisture level and comprising oil-containing microbes, with at least one grinding agent capable of absorbing oil); however, advantageously, the disruption occurs without requiring a liquid carrier. Furthermore, the presence of the grinding agent within the solid pellets appears to facilitate high levels of oil extraction. And, since the pellets remain durable throughout the extraction process, this aids operability and cycle time.

Extracted oil compositions comprising at least one PUFA, such as EPA (or derivatives thereof), will have well known clinical and pharmaceutical value. See, e,g., U.S. Pat. Appl. Pub. No. 2009-0093543 A1. For example, lipid compositions comprising PUFAs may be used as dietary substitutes, or supplements, particularly infant formulas, for patients undergoing intravenous feeding or for preventing or treating malnutrition. Alternatively, the purified PUFAs (or derivatives thereof) may be incorporated into cooking oils, fats or margarines formulated so that in normal use the recipient would receive the desired amount for dietary supplementation. The PUFAs may also be incorporated into infant formulas, nutritional supplements or other food products and may find use as anti-inflammatory or cholesterol lowering agents. Optionally, the compositions may be used for pharmaceutical use, either human or veterinary.

Supplementation of humans or animals with PUFAs can result in increased levels of the added PUFAs, as well as their metabolic progeny. For example, treatment with EPA can result not only in increased levels of EPA, but also downstream products of EPA such as eicosanoids (i.e., prostaglandins, leukotrienes, thromboxanes), DPAn-3 and DHA. Complex regulatory mechanisms can make it desirable to combine various PUFAs, or add different conjugates of PUFAs, in order to prevent, control or overcome such mechanisms to achieve the desired levels of specific PUFAs in an individual.

Alternatively, PUFAs, or derivatives thereof, can be utilized in the synthesis of animal and aquaculture feeds, such as dry feeds, semi-moist and wet feeds, since these formulations generally require at least 1-2% of the nutrient composition to be omega-3 and/or omega-6 PUFAs

### EXAMPLES

The present invention is further defined in the following examples. It should be understood that these examples, while indicating preferred embodiments of the invention, are given by way of illustration only. From the above discussion and these examples, one skilled in the art can ascertain the essential characteristics of this invention, and without departing from the spirit and scope thereof, can make various changes and modifications of the invention to adapt it to various uses and conditions.

The following abbreviations are used:

“HPLC” is High Performance Liquid Chromatography, “ASTM” is American Society for Testing And Materials, “C” is Celsius, “kPa” is kiloPascal, “mm” is millimeter, “μm” is micrometer, “4” is microliter, “mL” is milliliter, “L” is liter, “min” is minute, “mM” is millimolar, “mTorr” is milliTorr, “cm” is centimeter, “g” is gram, “wt” is weight, “h” or “hr” is hour, “temp” or “T” is temperature, “SS” is stainless steel, “in” is inch, “i.d.” is inside diameter, and “o.d.” is outside diameter.

### Materials

### Biomass Preparation

Described below are strains of Yarrowia lipolytica yeast producing various amounts of microbial oil comprising PUFAs. Biomass was obtained in a 2-stage fed-batch fermentation process, and then subjected to downstream processing, as described below.

Yarrowia lipolytica Strains:

The yeast biomass used in Comparative Examples C₁-C₄ and Examples 1 and 2 herein utilized Y. lipolytica strain Y8672. The generation of strain Y8672 is described in U.S. Pat. Appl. Pub. No. 2010-0317072-A1. Strain Y8672, derived from Y. lipolytica ATCC #20362, was capable of producing about 61.8% EPA relative to the total lipids via expression of a delta-9 elongase/delta-8 desaturase pathway.

The final genotype of strain Y8672 with respect to wild type Y. lipolytica ATCC #20362 was Ura+, Pex3−, unknown 1−, unknown 2−, unknown 3−, unknown 4−, unknown 5−, unknown 6−, unknown 7−, unknown 8−, Leu+, Lys+, YAT1::ME3S::Pex16, GPD::ME3S::Pex20, GPD::FmD12::Pex20, YAT1::FmD12::Oct, EXP1::FmD12S::ACO, GPAT::EgD9e::Lip2, FBAINm::EgD9eS::Lip2, EXP1::EgD9eS::Lip1, YAT1::EgD9eS::Lip2, FBAINm::EgD8M::Pex20, FBAIN::EgD8M::Lip1, EXP1::EgD8M::Pex16, GPD::EaD8S::Pex16 (2 copies), YAT1::E389D9eS/EgD8M::Lip1, YAT1::EgD9eS/EgD8M::Aco, FBAIN::EgD5SM::Pex20, YAT1::EgD5SM::Aco, GPM::EgD5SM::Oct, EXP1::EgD5M::Pex16, EXP1::EgD5SM::Lip1, YAT1::EaD5SM::Oct, YAT1::PaD17S::Lip1, EXP1::PaD17::Pex16, FBAINm::PaD17::Aco, GPD::YICPT1::Aco, and YAT1::MCS::Lip1. The structure of the above expression cassettes are represented by a simple notation system of “X::Y::Z”, wherein X describes the promoter fragment, Y describes the gene fragment, and Z describes the terminator fragment, which are all operably linked to one another. Abbreviations are as follows: FmD12 is a Fusarium moniliforme delta-12 desaturase gene [U.S. Pat. No. 7,504,259]; FmD12S is a codon-optimized delta-12 desaturase gene, derived from Fusarium moniliforme [U.S. Pat. No. 7,504,259]; MESS is a codon-optimized C_{16/18} elongase gene, derived from Mortierella alpina [U.S. Pat. No. 7,470,532]; EgD9e is a Euglena gracilis delta-9 elongase gene [U.S. Pat. No. 7,645,604]; EgD9eS is a codon-optimized delta-9 elongase gene, derived from Euglena gracilis [U.S. Pat. No. 7,645,604]; EgD8M is a synthetic mutant delta-8 desaturase gene [U.S. Pat. No. 7,709,239], derived from Euglena gracilis [U.S. Pat. No. 7,256,033]; EaD8S is a codon-optimized delta-8 desaturase gene, derived from Euglena anabaena [U.S. Pat. No. 7,790,156]; E389D9eS/EgD8M is a DGLA synthase created by linking a codon-optimized delta-9 elongase gene (“E389D9eS”), derived from Eutreptiella sp. CCMP389 delta-9 elongase (U.S. Pat. No. 7,645,604) to the delta-8 desaturase “EgD8M” (supra) [U.S. Pat. Appl. Pub. No. 2008-0254191-A1]; EgD9ES/EgD8M is a DGLA synthase created by linking the delta-9 elongase “EgD9eS” (supra) to the delta-8 desaturase “EgD8M” (supra) [U.S. Pat. Appl. Pub. No. 2008-0254191-A1]; EgD5M and EgD5SM are synthetic mutant delta-5 desaturase genes [U.S. Pat. App. Pub. 2010-0075386-A1], derived from Euglena gracilis [U.S. Pat. No. 7,678,560]; EaDSSM is a synthetic mutant delta-5 desaturase gene [U.S. Pat. App. Pub. 2010-0075386-A1], derived from Euglena anabaena [U.S. Pat. No. 7,943,365]; PaD17 is a Pythium aphanidermatum delta-17 desaturase gene [U.S. Pat. No. 7,556,949]; PaD17S is a codon-optimized delta-17 desaturase gene, derived from Pythium aphanidermatum [U.S. Pat. No. 7,556,949]; YICPT1 is a Yarrowia lipolytica diacylglycerol cholinephosphotransferase gene [U.S. Pat. No. 7,932,077]; and, MCS is a codon-optimized malonyl-CoA synthetase gene, derived from Rhizobium leguminosarum by. viciae 3841 [U.S. Pat. App. Pub. 2010-0159558-A1].

For a detailed analysis of the total lipid content and composition in strain Y8672, a flask assay was conducted wherein cells were grown in 2 stages for a total of 7 days. Based on analyses, strain Y8672 produced 3.3 g/L dry cell weight [“DCW”], total lipid content of the cells was 26.5 [“TFAs % DCW”], the EPA content as a percent of the dry cell weight [“EPA % DCW”] was 16.4, and the lipid profile was as follows, wherein the concentration of each fatty acid is as a weight percent of TFAs [“% TFAs”]: 16:0 (palmitate)-2.3, 16:1 (palmitoleic acid)—0.4, 18:0 (stearic acid)—2.0, 18:1 (oleic acid)—4.0, 18:2 (LA)—16.1, ALA—1.4, EDA—1.8, DGLA—1.6, ARA—0.7, ETrA—0.4, ETA—1.1, EPA—61.8, other—6.4.

In contrast, the yeast biomass used in Comparative Examples C₅-C₆ and Examples 3 and 10 herein utilized Y. lipolytica strain Y9502. The generation of strain Y9502 is described in U.S. Pat. Appl. Pub. No. 2010-0317072-A1, hereby incorporated herein by reference in its entirety. Strain Y9502, derived from Y. lipolytica ATCC #20362, was capable of producing about 57.0% EPA relative to the total lipids via expression of a delta-9 elongase/delta-8 desaturase pathway.

The final genotype of strain Y9502 with respect to wildtype Y. lipolytica ATCC #20362 was Ura+, Pex3−, unknown 1−, unknown 2−, unknown 3−, unknown 4−, unknown 5−, unknown6−, unknown 7−, unknown 8−, unknown9−, unknown 10-, YAT1::ME3S::Pex16, GPD::ME3S::Pex20, YAT1::ME3S::Lip1, FBAINm::EgD9eS::Lip2, EXP1::EgD9eS::Lip1, GPAT::EgD9e::Lip2, YAT1::EgD9eS::Lip2, FBAINm::EgD8M::Pex20, EXP1::EgD8M::Pex16, FBAIN::EgD8M::Lip1, GPD::EaD8S::Pex16 (2 copies), YAT1::E389D9eS/EgD8M::Lip1, YAT1::EgD9eS/EgD8M::Aco, FBAINm::EaD9eS/EaD8S::Lip2, GPD::FmD12::Pex20, YAT1::FmD12::Oct, EXP1::FmD12S::Aco, GPDIN::FmD12::Pex16, EXP1::EgD5M::Pex16, FBAIN::EgD5SM::Pex20, GPDIN::EgD5SM::Aco, GPM::EgD5SM::Oct, EXP1::EgD5SM::Lip1, YAT1::EaD5SM::Oct, FBAINm::PaD17::Aco, EXP1::PaD17::Pex16, YAT1::PaD17S::Lip1, YAT1::YICPT::Aco, YAT1::MCS::Lip1, FBA::MCS::Lip1, YAT1::MaLPAAT1S::Pex16. Abbreviations not previously defined are as follows: EaD9eS/EgD8M is a DGLA synthase created by linking a codon-optimized delta-9 elongase gene (“EaD9eS”), derived from Euglena anabaena delta-9 elongase [U.S. Pat. No. 7,794,701] to the delta-8 desaturase “EgD8M” (supra) [U.S. Pat. Appl. Pub. No. 2008-0254191-A1]; and, MaLPAAT1S is a codon-optimized lysophosphatidic acid acyltransferase gene, derived from Mortierella alpina [U.S. Pat. No. 7,879,591].

For a detailed analysis of the total lipid content and composition in strain Y9502, a flask assay was conducted wherein cells were grown in 2 stages for a total of 7 days. Based on analyses, strain Y9502 produced 3.8 g/L dry cell weight [“DCW”], total lipid content of the cells was 37.1 [“TFAs % DCW”], the EPA content as a percent of the dry cell weight [“EPA % DCW”] was 21.3, and the lipid profile was as follows, wherein the concentration of each fatty acid is as a weight percent of TFAs [“% TFAs”]: 16:0 (palmitate)-2.5, 16:1 (palmitoleic acid)—0.5, 18:0 (stearic acid)—2.9, 18:1 (oleic acid)—5.0, 18:2 (LA)—12.7, ALA—0.9, EDA—3.5, DGLA—3.3, ARA—0.8, ETrA—0.7, ETA—2.4, EPA—57.0, other—7.5.

Fermentation:

Inocula were prepared from frozen cultures of Yarrowia lipolytica in a shake flask. After an incubation period, the culture was used to inoculate a seed fermentor. When the seed culture reached an appropriate target cell density, it was then used to inoculate a larger fermentor. The fermentation is a 2-stage fed-batch process. In the first stage, the yeast were cultured under conditions that promote rapid growth to a high cell density; the culture medium comprised glucose, various nitrogen sources, trace metals and vitamins. In the second stage, the yeast were starved for nitrogen and continuously fed glucose to promote lipid and PUFA accumulation. Process variables including temperature (controlled between 30-32° C.), pH (controlled between 5-7), dissolved oxygen concentration and glucose concentration were monitored and controlled per standard operating conditions to ensure consistent process performance and final PUFA oil quality.

One of skill in the art of fermentation will know that variability will occur in the oil profile of a specific Yarrowia strain, depending on the fermentation run itself, media conditions, process parameters, scale-up, etc., as well as the particular time-point in which the culture is sampled (see, e.g., U.S. Pat. Appl. Pub. No. 2009-0093543-A1).

Downstream Processing:

Antioxidants were optionally added to the fermentation broth prior to processing to ensure the oxidative stability of the EPA oil. The yeast biomass was dewatered and washed to remove salts and residual medium, and to minimize lipase activity. Either drum-drying (typically with 80 psig steam) or spray-drying was then performed, to reduce moisture level to less than 5% to ensure oil stability during short term storage and transportation. The drum dried flakes, or spray dried powder having particle size distribution in range of about 10 to 100 micron, were used in the following Comparative Examples and Examples, as the initial “microbial biomass, comprising oil-containing microbes”.

Grinding Agents:

Celite 209 D-earth is available from Celite Corporation, Lompoc, Calif. Celatom MN-4 D-earth is available from EP Minerals, An Eagle Pitcher Company, Reno, Nev.

Other Materials:

All commercial reagents were used as received. All solvents used were HPLC grade. Acetyl chloride was 99+%. TLC plates and solvents were obtained from VWR (West Chester, Pa.). HPLC or SCF grade carbon dioxide was obtained from MG Industries (Malvern, Pa.).

### Methods

### Twin Screw Extrusion Method

Twin screw extrusion was used in disrupting dried yeast biomass and preparing disrupted biomass mix with grinding agents.

Dried yeast is fed into an extruder, preferably a twin screw extruder with a length, normally 21-39 L/D, suitable for accomplishing the operations described below (although this particular L/D ratio should not be considered a limitation herein). The first section of the extruder is used to feed and transport the materials. The second section is a compaction zone designed to compact and compress the feed using bushing elements with progressively shorter pitch length. After the compaction zone, a compression zone follows which serves to impart most of the mechanical energy required for cell disruption. This zone is created using flow restriction either in the form of reverse screw elements or kneading elements. When preparing disrupted biomass, the grinding agent (e.g., D-earth) is co-fed with the microbial biomass feed so that both go through the compression/compaction zone, thus enhancing disruption levels. Following the compression zone, the binding agent (e.g., water/sucrose solution) is added through a liquid injection port and mixed in subsequent mixing sections comprised of various combinations of mixing elements. The final mixture (i.e., the “fixable mix”) is discharged through the last barrel which is open at the end, thus producing little or no backpressure in the extruder. The fixable mix is then fed into a dome granulator and either a vibrating or a fluidized bed drier. This results in pelletized material (i.e., solid pellets) suitable for downstream oil extraction.

### SCF Extraction With CO₂

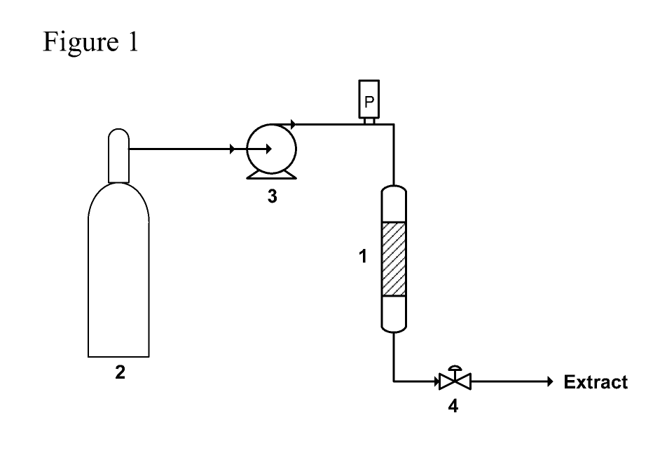

Supercritical CO₂ extraction of yeast samples in the examples below was conducted in a custom high-pressure extraction apparatus illustrated in the flowsheet of FIG. 1. In general, dried and mechanically disrupted yeast cells (free flowing or pelletized) were charged to an extraction vessel (1) packed between plugs of glass wool, flushed with CO₂, and then heated and pressurized to the desired operating conditions under CO₂ flow. The 89-ml extraction vessels were fabricated from 316 SS tubing (2.54 cm o.d.×1.93 cm i.d.×30.5 cm long) and equipped with a 2-micron sintered metal filter on the effluent end of the vessel. The extraction vessel was installed inside of a custom machined aluminum block equipped with four calrod heating cartridges which were controlled by an automated temperature controller. The CO₂ was fed as a liquid directly from a commercial cylinder (2) equipped with an eductor tube and was metered with a high-pressure positive displacement pump (3) equipped with a refrigerated head assembly (Jasco Model PU-1580-CO₂). Extraction pressure was maintained with an automated back pressure regulator (4) (Jasco Model BP-1580-81) which provided a flow restriction on the effluent side of the vessel, and the extracted oil sample was collected in a sample vessel while simultaneously venting the CO₂ solvent to the atmosphere.

Reported oil extraction yields from the yeast samples were determined gravimetrically by measuring the mass loss from the sample during the extraction. Thus, the reported extracted oil comprises microbial oil and moisture associated with the solid pellets.

### Examples

### Comparative Examples C1, C2A, C2B, Example 1, Example 2 And Comparative Examples C3 And C4

### Comparison of Means to Create a Disrupted Biomass Mix from Drum-Dried Flakes of Yarrowia lipolytica

Comparative Examples C1, C2A, C2B, C3 and C4 and Examples 1 and 2 describe a series of comparative tests performed to optimize disruption of drum dried flakes of yeast (i.e., Yarrowia lipolytica strain Y8672). Specifically, hammer milling with and without the addition of grinding agent was examined, as well as use of either a single screw or twin screw extruder. Results are compared based on the total free microbial oil and disruption efficiency of the microbial cells, as well as the total extraction yield (based on supercritical CO₂ extraction).

### Comparative Example C1

### Hammer-Milled Yeast Powder Without Grinding Agent

Drum dried flakes of yeast (Yarrowia lipolytica strain Y8672) biomass containing 24.2% total oil (dry weight) were hammer-milled (Mikropul Bantam mill at a feed rate of 12 Kg/h) at ambient temperature using a jump-gap separator at 16,000 rpm with three hammers to provide milled powder. Particle size of the milled powder was d10=3 μm; d50=16 μm and d90=108 μm, analyzed suspended in water using Frauenhofer laser diffraction.

### Comparative Example C2A

### Hammer-Milled Yeast Powder With Grinding Agent and Air Mill Mixing

The hammer-milled yeast powder provided by Comparative Example C1 (833 g) was mixed with Celite 209 diatomaceous earth (D-earth) (167 g) in an air (jet) mill (Fluid Energy Jet-o-mizer 0101, at a feed rate of 6 Kg/h) for about 20 min at ambient temperature.

### Comparative Example C2B

### Hammer-Milled Yeast Powder With Grinding Agent and Manual Mixing

Hammer-milled yeast powder provided by Comparative Example C1 (833 g) was mixed manually with Celite 209 D-earth (167 g) in a plastic bag.

### Example 1

### Hammer Milled Yeast Powder With Grinding Agent, Manual Mixing, and Single Screw Extruder

The hammer-milled yeast powder with D-earth from Comparative Example C2B (1000 g) was mixed with a 17.6 wt % aqueous sucrose solution (62.5 g sucrose in 291.6 g water) in a Hobart mixer for about 2.5 min and then extruded (50-200 psi, torque not exceeding 550 in-lbs; 40° C. or less extrudate temperature) through a single screw dome granulator having 1 mm orifices. The extrudate was dried in a fluid bed dryer to a bed temperature of 50° C. using fluidizing air controlled at 65° C. to provide non-sticky pellets (815 g, having dimensions of 2 to 8 mm length and about 1 mm diameter) having 3.9% water remaining after about 14 min.

### Example 2

### Hammer Milled Yeast Powder With Grinding Agent, Air Mill Mixing, And Single Screw Extruder

The hammer milled yeast powder with D-earth from Comparative Example C2A (1000 g) was processed according to Example 1 to provide pellets (855 g, having dimensions of 2 to 8 mm length and about 1 mm diameter) having 6.9% water remaining after about 10 min.

### Comparative Example C3

### Hammer Milled Yeast Powder Without Grinding Agent and with Twin Screw Extruder

The hammer milled yeast powder provided from Comparative Example C1 was fed at 2.3 kg/hr to an 18 mm twin screw extruder (Coperion Werner Pfleiderer ZSK-18 mm MC, Stuttgart, Germany) operating with a 10 kW motor and high torque shaft, at 150 rpm and % torque range of 66-68 to provide a disrupted yeast powder cooled to 26° C. in a final water cooled barrel.

### Comparative Example C4

### Yeast Powder without Grinding Agent and with Twin Screw Extruder

Drum dried flakes of yeast (Yarrowia lipolytica strain Y8672) biomass containing 24.2% total oil were fed at 2.3 kg/hr to an 18 mm twin screw extruder (Coperion Werner Pfleiderer ZSK-18 mm MC) operating with a 10 kW motor and high torque shaft, at 150 rpm and % torque range of 71-73 to provide a disrupted yeast powder cooled to 23° C. in a final water cooled barrel.

### Comparison of Free Microbial Oil and Disruption Efficiency in Disrupted Yeast Powder

The free microbial oil and disruption efficiency was determined in the disrupted yeast powders of Examples 1 and 2, and Comparative Examples C1-C4 according to the following method. Specifically, free oil and total oil content of extruded biomass samples were determined using a modified version of the method reported by Tröeng (J. Amer. Oil Chemists Soc., 32:124-126 (1955)). In this method, a sample of the extruded biomass was weighed into a stainless steel centrifuge tube with a measured volume of hexane. Several chrome steel ball bearings were added if total oil was to be determined. The ball bearings were not used if free oil was to be determined. The tubes were then capped and placed on a shaker for 2 hours. The shaken samples were centrifuged, the supernatant was collected and the volume measured. The hexane was evaporated from the supernatant first by rotary film evaporation and then by evaporation under a stream of dry nitrogen until a constant weight was obtained. This weight was then used to calculate the percentage of free or total oil in the original sample. The oil content is expressed on a percent dry weight basis by measuring the moisture content of the sample, and correcting as appropriate.

The percent disruption efficiency (i.e., the percent of cells walls that have been fractured during processing) was quantified by optical visualization.

Table 4 summarizes the yeast cell disruption efficiency data for Examples 1 and 2, and Comparative Examples C1-C4, and reveals the following:

Comparative Example C1 shows that Hammer milling in the absence of grinding agent results in 33% disruption of the yeast cells.

Comparative Example C2A shows that air jet milling of Hammer-milled yeast in the presence of grinding agent increases the disruption of the yeast cells to 62%.

Example 1 shows that further mixing of Hammer-milled yeast (from Comparative Example C1) in a Hobart single-screw mixer in the presence of grinding agent increases the disruption of the yeast cells to 38%.

Example 2 shows that further mixing of air-milled and Hammer-milled yeast with grinding agent (from Comparative Example C2A) in a Hobart single-screw mixer increases the disruption of the yeast cells to 57%.

Comparative Examples C3 and C4 show that in the absence of grinding agent and with or without Hammer-milling (respectively), using twin screw extrusion with a compression zone, the yeast cell disruption was greater than 80%.

TABLE 4
Comparison Of Yeast Cell Disruption Efficiency

| | Free Oil | Disruption |
|---|---|---|
| Example | % DWT | Efficiency, % |
| C1 | 8 | 33 |
| C2A* | 12.6 | 62 |
| 1* | 9.2 | 38 |
| 2* | 13.8 | 57 |
| C3 | 19.6 | 82 |
| C4 | 21 | 87 |

*The free oil liberated is normalized using the actual weight fraction of biomass in the pellet in Example 1, Example 2 and Comparative Example C2A.

### SCF Extraction

The extraction vessel was charged with approximately 25 g (yeast basis) of disrupted yeast biomass from Comparative Examples C1, C2A and C4, respectively. The yeast were flushed with CO₂, then heated to approximately 40° C. and pressurized to approximately 311 bar. The yeast were extracted at these conditions at a flow rate of 4.3 g/min CO₂ for approximately 6.7 hr, giving a final solvent-to-feed (S/F) ratio of about 75 g CO₂/g yeast. Extraction yields are reported in Table 5.

The data show that higher cell disruption leads to significantly higher extraction yields, measured as the weight percent of crude extracted oil.

TABLE 5
Comparison Of Cell Disruption Efficiency And Oil Extraction

| | | | | | | | Ex- |
|---|---|---|---|---|---|---|---|
| | Yeast | Cell | | | | S/F | tracted |
| | Charge | disruption | | Pres- | | ratio (g | Oil |
| Exam- | (g Dry | efficiency | Temp. | sure | Time | CO₂/g | Yield |
| ple | weight) | (%) | (° C.) | (bar) | (hr) | yeast) | (wt %) |
| C1 | 25.1 | 33 | 40 | 310 | 6.6 | 74.7 | 7.5 |
| C2A | 25.0 | 52 | 40 | 311 | 6.8 | 76.7 | 8.9 |
| C4 | 25.2 | 87 | 41 | 310 | 6.7 | 74.4 | 18.8 |

### Comparative Examples C5A, C5B, C5C, C6A, C6B And C6C

### Comparison Of Means to Create a Disrupted Biomass Mix from Yarrowia lipolytica

Comparative Examples C5A, C5B, C5C, C6A, C6B and C6C describe a series of comparative tests performed to prepare disrupted yeast powder, wherein the initial microbial biomass was either drum dried flakes or spray-dried powder of yeast, mixed with or without a grinding agent in a twin-screw extruder.

In each of Comparative Examples C5A, C5B, C5C, C6A, C6B and C6C, the initial yeast biomass was from Yarrowia lipolytica strain Y9502, having a moisture level of 2.8% and containing approximately 36% total oil. The dried yeast flakes or powder (with or without grinding agent) were fed to an 18 mm twin screw extruder (Coperion Werner Pfleiderer ZSK-18 mm MC) operating with a 10 kW motor and high torque shaft, at 150 rpm. The resulting disrupted yeast powder was cooled in a final water cooled barrel.

The disrupted yeast powder prepared in Comparative Examples C5A, C5B, C5C, C6A, C6B and C6C was then subjected to supercritical CO₂ extraction and total extraction yields were compared.

### Comparative Example C5A

### Drum-Dried Yeast Flakes without Grinding Agent

Drum dried flakes of yeast biomass were fed at 2.3 kg/hr to the twin screw extruder operating with a % torque range of 34-35. The disrupted yeast powder was cooled to 27° C.

### Comparative Example C5B

### Drum-Dried Yeast Flakes with Grinding Agent

92.5 parts of drum dried flakes of yeast biomass were premixed in a bag with 7.5 parts of Celite 209 D-earth. The resultant dry mix was fed at 2.3 kg/hr to the twin screw extruder operating with a % torque range of 44-47. The disrupted yeast powder was cooled to 29° C.

### Comparative Example C5C

### Drum-Dried Yeast Flakes with Grinding Agent

85 parts of drum dried flakes of yeast biomass were premixed in a bag with 15 parts of Celite 209 D-earth. The resultant dry mix was fed at 2.3 kg/hr to the twin screw extruder operating with a % torque range of 48-51. The disrupted yeast powder was cooled to 29° C.

### Comparative Example C6A

### Spray-Dried Yeast Powder without Grinding Agent

Spray dried powder of yeast biomass were fed at 1.8 kg/hr to the twin screw extruder operating with a % torque range of 33-34. The disrupted yeast powder was cooled to 26° C.

### Comparative Example C6B

### Spray-Dried Yeast Powder with Grinding Agent

92.5 parts of spray dried powder of yeast biomass were premixed in a bag with 7.5 parts of Celite 209 D-earth. The resultant dry mix was fed at 1.8 kg/hr to the twin screw extruder operating with a % torque range of 37-38. The disrupted yeast powder was cooled to 26° C.

### Comparative Example C6C

### Spray-Dried Yeast Powder with Grinding Agent

85 parts of spray dried powder of yeast biomass were premixed in a bag with 15 parts of D-earth (Celite 209). The resultant dry mix was fed at 1.8 kg/hr to the twin screw extruder operating with a % torque range of 38-39. The disrupted yeast powder was cooled to 27° C.

### SCF Extraction

The extraction vessel was charged with 11.7 g (yeast basis) of disrupted yeast biomass from Comparative Examples C5A, C5B, C5C, C6A, C6B and C6C, respectively. The yeast was flushed with CO₂, then heated to approximately 40° C. and pressurized to approximately 311 bar. The yeast samples were extracted at these conditions at a flow rate of 4.3 g/min CO₂ for 3.2 hr, giving a final solvent-to-feed (S/F) ratio of approximately 76.6 g CO₂/g yeast. Extraction yields for various formulations are reported in Table 6.

The data show that samples having D-earth as a grinding agent (i.e., Comparative Examples C5B, C5C, C6B and C6C) lead to higher extraction yields than those wherein D-earth was not present (i.e., Comparative Examples C5A and C6A).

TABLE 6
Comparison Of Oil Extraction Of Disrupted Yeast With And Without
Grinding Agent

| | Yeast | | | CO₂ | | S/F | |
|---|---|---|---|---|---|---|---|
| | Charge | | Pres- | Flow | | ratio | Extracted |
| Exam- | (g Dry | Temp. | sure | Rate | Time | (g CO₂/ | Oil Yield |
| ple | weight) | (° C.) | (bar) | (g/min) | (hr) | g yeast) | (wt %) |
| C5A | 11.7 | 40 | 311 | 4.3 | 3.2 | 76.4 | 31.8 |
| C5B | 11.7 | 41 | 312 | 4.3 | 3.2 | 76.6 | 35.4 |
| C5C | 11.7 | 40 | 312 | 4.3 | 3.2 | 76.7 | 35.1 |
| C6A | 11.7 | 40 | 311 | 4.3 | 3.2 | 76.4 | 30.5 |
| C6B | 11.7 | 40 | 311 | 4.3 | 3.2 | 76.6 | 37.9 |
| C6C | 11.7 | 40 | 311 | 4.3 | 3.2 | 76.7 | 38.8 |

### Examples 3, 4, 5, 6, 7, 8, 9 and 10

### Comparison of Means to Create Solid Pellets from Yarrowia lipolytica

Examples 3-10 describe a series of comparative tests performed to mix spray dried powder or drum-dried flakes of yeast biomass with a grinding agent and binding agent, to provide solid pellets.

In each of Examples 3-10, the initial yeast biomass was from Yarrowia lipolytica strain Y9502, having a moisture level of 2.8% and containing approximately 36% total oil. Following preparation of solid pellets, approximately 1 mm diameter×2 to 8 mm in length, the pellets were subjected to supercritical CO₂ extraction and total extraction yields were compared. Mechanical compression properties and attrition resistance of the solid pellets were also analyzed.

### Example 3

85 parts of spray dried powder of yeast biomass were premixed in a bag with 15 parts of Celatom MN-4 D-earth. The resultant dry mix was fed at 2.3 kg/hr to an 18 mm twin screw extruder (Coperion Werner Pfleiderer ZSK-18 mm MC). Along with the dry feed, a water/sugar solution made of 14 parts water and 5.1 parts sugar was injected after the disruption zone of the extruder at a flow-rate of 8.2 ml/min. The extruder was operating with a 10 kW motor and high torque shaft, at 150 rpm and % torque range of 58-60 to provide a disrupted yeast powder cooled to 24° C. in a final water cooled barrel.

The fixable mix was then fed into a MG-55 LCI Dome Granulator assembled with 1 mm hole diameter by 1 mm thick screen and set to 70 RPM. Extrudates were formed at 67.5 kg/hr and a steady 2.7 amp current. The sample was dried in a Sherwood Dryer for 10 min to provide solid pellets having a final moisture level of 7.1%.

### Example 4

A fixable mix prepared according to Example 3 was passed through a granulator at 45 RPM. Extrudates were formed at 31.7 kg/hr and dried in a Sherwood Dryer for 10 min to provide solid pellets having a final moisture level of 8.15%.

### Example 5

A fixable mix prepared according to Example 3 was passed through a granulator at 90 RPM. Extrudate pellets were dried in a MDB-400 Fluid Bed Dryer for 15 min to provide solid pellets having a final moisture level of 4.53%.

### Example 6

85 parts of spray dried powder of yeast biomass were premixed in a bag with 15 parts of Celatom MN-4 D-earth. The resultant dry mix was fed at 2.3 kg/hr to an 18 mm twin screw extruder (Coperion Werner Pfleiderer ZSK-18 mm MC) operating with a 10 kW motor and high torque shaft, at 150 rpm and % torque range of 70-74 to provide a disrupted yeast powder cooled to 31° C. in a final water cooled barrel.

The disrupted yeast powder was then mixed in a Kitchen Aid mixer with a 22.6% solution of sucrose and water (i.e., 17.5 parts water and 5.1 parts sugar). The total mix time was 4.5 min with the solution added over the first 2 min.

The fixable mix was fed to a MG-55 LCI Dome Granulator assembled with 1 mm hole diameter by 1 mm thick screen and set to 70 RPM. Extrudates were formed at 71.4 kg/hr and a steady 2.7 amp current. The sample was dried in a Sherwood Dryer for a total of 20 min to provide solid pellets having a final moisture level of 6.5%.

### Example 7

Disrupted yeast powder prepared according to Example 6 was placed in a KDHJ-20 Batch Sigma Blade Kneader with a 22.6% solution of sucrose and water (i.e., 17.5 parts water and 5.1 parts sugar). The total mix time was 3.5 min with the solution added over the first 2 min.

The fixable mix was fed to a MG-55 LCI Dome Granulator assembled with 1 mm hole diameter by 1 mm thick screen and set to 90 RPM. Extrudates were formed at 47.5 kg/hr and a steady 2.3 amp current. The sample was dried in a Sherwood Dryer for a total of 15 min to provide solid pellets having a final moisture level of 7.4%.

### Example 8

Drum dried flakes of yeast biomass were fed at 1.8 kg/hr to an 18 mm twin screw extruder (Coperion Werner Pfleiderer ZSK-18 mm MC) operating with a 10 kW motor and high torque shaft, at 150 rpm and % torque range of 38-40 to provide a disrupted yeast powder cooled to 30° C. in a final water cooled barrel.

The disrupted yeast powder (69.5 parts) was mixed in a Kitchen Aid mixer with 12.2% Celite 209 D-earth (12.2 parts) and an aqueous sucrose solution (18.3 parts) made from a 3.3 ratio of water to sugar. The total mix time was 4.5 min with the solution added over the first 2 min.

The fixable mix was fed to a MG-55 LCI Dome Granulator assembled with 1 mm hole diameter by 1 mm thick screen and set to 90 RPM. Extrudates were formed at 68.2 kg/hr and a steady 2.5 amp current. The sample was dried in a Sherwood Dryer for a total of 15 min to provide solid pellets having a final moisture level of 6.83%.

### Example 9

Drum dried flakes of yeast biomass (85 parts) were premixed in a bag with 15 parts of Celite 209 D-earth. The resultant dry mix was fed at 2.3 kg/hr to an 18 mm twin screw extruder (Coperion Werner Pfleiderer ZSK-18 mm MC). Along with the dry feed, a water/sugar solution made of 14 parts water and 5.1 parts sugar was injected after the disruption zone of the extruder at a flowrate of 8.2 ml/min. The extruder was operating with a 10 kW motor and high torque shaft, at 150 rpm and % torque range of 61-65 to provide a disrupted yeast powder cooled to 25° C. in a final water cooled barrel.

The fixable mix was then fed into a MG-55 LCI Dome Granulator assembled with 1 mm hole diameter by 1 mm thick screen and set to 90 RPM. Extrudates were formed at 81.4 kg/hr and a steady 2.5 amp current. The sample was dried in a Sherwood Dryer for 15 min to provide solid pellets having a final moisture level of 8.3%.

### Example 10

Drum dried flakes of yeast biomass (85 parts) were premixed in a bag with 15 parts of Celatom NM-4 D-earth. The resultant dry mix was fed at 4.6 kg/hr to an 18 mm twin screw extruder (Coperion Werner Pfleiderer ZSK-18 mm MC). Along with the dry feed, a water/sugar solution made of 14 parts water and 5.1 parts sugar was injected after the disruption zone of the extruder at a flowrate of 8.2 ml/min. The extruder was operating with a 10 kW motor and high torque shaft, at 300 rpm and % torque of about 34 to provide a disrupted yeast powder.

The fixable mix was then fed into a MG-55 LCI Dome Granulator assembled with 1 mm hole diameter by 1 mm thick screen and set to 90 RPM. Extrudate was formed at 81.4 kg/hr and a steady 2.5 amp current. The sample was dried in a Sherwood Dryer for 15 min to provide solid pellets.

### Compression Testing and Attrition Resistance of Solid Pellets

Compression testing was performed as follows. The testing apparatus and protocol described in ASTM Standard D-6683 was used to assess the response of solid pellets to external loads, such as that imposed by a gas pressure gradient. In the test, the volume of a known mass is measured as a function of a mechanically applied compaction stress. A semi-log graph of the results typically is a straight line with a slope, β, reflecting the compression of the sample. Higher values of β reflect greater compression. This compression can be indicative of particle breakage, which would lead to undesirable segregation and gas flow restriction in processing.

At the conclusion of the ASTM test, the load was maintained on the pellets an additional 2 hrs, simulating extended processing time. Creep, measured after 2 hrs, is a further indication of the likelihood of the solid pellet to deform. Lower creep indicates less deformation.

The test cell containing the sample was then inverted, and the pellet sample was poured out. If necessary, the cell was gently tapped to release the contents. The ease of emptying the cell and the resultant texture (i.e., loose or agglomerated) of the pellets was noted.

The texture after the test is a qualitative observation of how hard it was to empty the test cell used in the previous measurements. The most desirable samples poured out immediately, while some required increasing amounts of tapping, and may have fallen out in large chunks (i.e., less desirable).

To determine attrition resistance, solid pellets (10 g) previously compressed in the Compression Testing ASTM test were then transferred to a 3″ diameter, 500 micron sieve. The sieve was tapped by hand to remove any initial fragments of pellets smaller than 500 microns. The net weight of remaining pellets was noted. Then three cylindrical grinding media beads, each 0.50″ diameter by 0.50″ thick, weighing 5.3 grams each, were added to the sieve. The sieve was placed in an automatic sieve shaker (Gilson Model SS-3, with a setting of “8”, with automatic tapping “on”) and shaken for periods of 2, 5 or 10 min. The grinding media beads repeatedly strike the pellets from random angles. After shaking, the pan under the sieve was weighed to determine the amount of material that had been attrited and had fallen through the sieve. This test is intended to simulate very rough handling of the pellets after the oil extraction process.

Solid pellets from Examples 3-10, respectively, were analyzed to determine their compression properties and attrition resistance. Results are tabulated below in Table 7.

TABLE 7
Mechanical Compression And Attrition Of Solid Pellets

| | Loose | | Creep | | Attrition | |
|---|---|---|---|---|---|---|
| | Bulk | | after 2 hr | | In sieving | |
| Exam- | Density | Compression | at 1994 | Texture | 2 min | 10 min |
| ple | lb/ft³ | Exponent β | lb/ft² (%) | after test | (%) | (%) |
| 10 | 28.98 | 0.06857 | 12.78 | Puck, | 2.9 | 11.8 |
| | | | | breaks | | |
| | | | | into 5 | | |
| | | | | pieces | | |
| 3 | 31.27 | 0.05335 | 6.95 | No puck | 20.5 | 99.0 |
| 4 | 31.85 | 0.05966 | 13.07 | Many | 8.8 | 47.4 |
| | | | | taps, 5 | | |
| | | | | pieces | | |
| 5 | 24.66 | 0.03928 | 4.10 | Two taps, | 8.8 | 42.1 |
| | | | | loose | | |
| 6 | 30.63 | 0.04746 | 8.34 | Few taps, | 10.0 | 48.7 |
| | | | | loose | | |
| 7 | 28.89 | 0.04347 | 3.11 | Few taps, | 9.1 | 43.1 |
| | | | | loose | | |
| 8 | 28.35 | 0.02976 | 0.00 | Loose | 5.2 | 22.4 |
| 9 | 31.66 | 0.07730 | 16.06 | Puck | 7.5 | 36.2 |

### SCF Extraction

The extraction vessel was charged with solid pellets (on a dry weight basis, as listed in Table 8) from Examples 3-9, respectively. The pellets were flushed with CO₂, then heated to about 40° C. and pressurized to approximately 311 bar. The pellets were extracted at these conditions at a flow rate of 4.3 g/min CO₂ for about 6.8 hr, giving a final solvent-to-feed (S/F) ratio of approximately 150 g CO₂/g yeast. In some Examples a second run was performed for an additional 4.8 hrs, such that the total time for extraction was 11.6 hr. The oil extraction yields and specific parameters used for extraction are listed in Table 8.

TABLE 8
Comparison Of Oil Extraction Of Solid Pellets

| | Yeast | | | CO₂ | | S/F | |
|---|---|---|---|---|---|---|---|
| | Charge | | Pres- | Flow | | ratio | Extracted |
| Exam- | (g Dry | Temp. | sure | Rate | Time | (g CO₂/ | Oil Yield |
| ple | weight) | (° C.) | (bar) | (g/min) | (hr) | g yeast) | (wt %) |
| 3 | 12.8 | 40 | 311 | 4.3 | 6.8 | 150 | 37.3 |
| 4 | 21.5^{b} | 40 | 312 | 4.3 | 11.6 | 151 | 39.3^{a} |
| 5 | 12.9 | 40 | 312 | 4.3 | 6.9 | 150 | 36.4 |
| 6 | 12.8 | 41 | 311 | 4.3 | 6.8 | 149 | 36.6 |
| 7 | 21.7^{b} | 40 | 312 | 4.3 | 11.6 | 150 | 37.4^{a} |
| 8 | 21.8^{b} | 40 | 311 | 4.3 | 11.6 | 150 | 31.0^{a} |
| 9 | 12.6 | 41 | 312 | 4.3 | 6.8 | 152 | 39.1 |

^{a}average result from two runs
^{b}sum of two runs

### Compression Testing And Attrition Resistance of Residual Pellets (Post-Extraction)

Following SCF extraction, the residual pellets from Examples 3-9, respectively, were analyzed to determine their compression properties and attrition resistance. Results are tabulated below in Table 9.

TABLE 9
Mechanical Compression And Attrition Of Residual Pellets (Post-
Extraction)

| | Loose | | Creep | | Attrition | |
|---|---|---|---|---|---|---|
| | Bulk | | after 2 hr | | In sieving | |
| | Density | Compression | at 1994 | Texture | 2 min | 5 min |
| Example | lb/ft³ | Exponent β | lb/ft² (%) | after test | (%) | (%) |
| 3 | 23.64 | 0.03352 | 0.75 | Loose | n/a | 73.0* |
| 4 | 23.50 | 0.02035 | 0.78 | One tap, | 12.0 | 28.6 |
| | | | | loose | | |
| 5 | 24.14 | 0.02636 | 0.71 | Loose | 10.9 | 26.4 |
| 6 | 24.66 | 0.02002 | 0.62 | Loose | 10.6 | 27.1 |
| 7 | 21.78 | 0.02897 | 0.98 | One tap, | 10.9 | 25.5 |
| | | | | loose | | |
| 8 | 21.84 | 0.02821 | 0.67 | Loose | 7.7 | 18.5 |
| 9 | 23.87 | 0.02246 | 0.58 | Loose | 10.1 | 22.3 |

*The expected attrition from 5 minutes of sieving was estimated by interpolating the results of a 2 minute test and a 6.5 minute test

Based on the above, it is concluded that the process described herein [i.e., comprising steps of (a) mixing a microbial biomass, having a moisture level and comprising oil-containing microbes, and at least one grinding agent capable of absorbing oil, to provide a disrupted biomass mix; (b) blending at least one binding agent with said disrupted biomass mix to provide a fixable mix capable of forming a solid pellet; and (c) forming said solid pellet from the fixable mix] can be successfully utilized to produce solid pellets comprising disrupted microbial biomass from Yarrowia lipolytica. Furthermore, the present Example demonstrates that the solid Y. lipolytica pellets can be extracted with a solvent (i.e., SCF extraction) to provide an extract comprising the microbial oil.

### Example 11

### Creation of Solid Pellets from Nannochloropsis Algae and Oil Extraction Thereof

The present example describes tests performed to demonstrate the applicability of the methodologies disclosed herein for use with a microbial biomass other than Yarrowia. Specifically, Nannochloropsis biomass was mixed with a grinding agent and binding agent, to provide solid pellets. These pellets were subjected to supercritical CO₂ extraction and total extraction yields were compared.

Kuehnle Agrosystems, Inc. (Honolulu, Hi.) provides a variety of axenic, unialgal stock algae for purchase. Upon request, they suggested algae strain KAS 604, comprising a Nannochloropsis species, as an appropriate microbial biomass having a lipid content of at least 20%. The biomass was grown under standard conditions (not optimizing conditions for oil content) and dried by Kuehnle Agrosystems, Inc. and then the microalgae powder was purchased for use below.

91.7 parts of microalgae powder were premixed in a bag with 8.3 parts of Celatom MN-4 D-earth. The resultant dry mix was fed at 0.91 kg/hr to an 18 mm twin screw extruder (Coperion Werner Pfleiderer ZSK-18 mm MC). Along with the dry feed, a 31% aqueous solution of sugar made of 10.9 parts water and 5.0 parts sugar was injected after the disruption zone of the extruder at a flow-rate of 2.5 mL/min. The extruder was operating with a 10 kW motor and high torque shaft, at 200 rpm and % torque range of 46-81 to provide a disrupted yeast powder cooled to 31° C. in a final water cooled barrel.

The fixable mix was then fed into a MG-55 LCI Dome Granulator assembled with 1.2 mm diameter holes by 1.2 mm thick screen and set to 20 RPM. Extrudates were formed at 20 kg/hr and a 6-7 amp current. The sample was dried in a Sherwood Dryer at 70° C. for 20 min to provide solid pellets having a final moisture level of 4.9%. The solid pellets, approximately 1.2 mm diameter×2 to 8 mm in length, were 82.1% algae, with the remainder of the composition being pelletization aids. The amount of total and free oil in the solid Nannochloropsis pellets was then determined and compared to the amount of oil extracted from the solid Nannochloropsis pellets by SCF.

### Determination of Total Oil Content in Solid Nannochloropsis Pellets

Specifically, total oil was determined on the pelletized sample by gently grinding it into a fine powder using a mortar and pestle, and then weighing aliquots (in triplicate) for analysis. The fatty acids in the sample (existing primarily as triglycerides) were converted to the corresponding methyl esters by reaction with acetyl chloride/methanol at 80° C. A C15:0 internal standard was added in known amounts to each sample for calibration purposes. Determination of the individual fatty acids was made by capillary gas chromatography with flame ionization detection (GC/FID). The sum of the fatty acids (expressed in triglyceride form) was 6.1%; this was taken to be the total oil content of the sample. After normalization, since the algae in the pellets represented only 82.1% of the total mass, the total oil content in the algae was determined to be 7.4% (i.e., 6.1% divided by 0.821).

The distribution of the individual fatty acids within the total oil sample is shown in the Table below.

TABLE 10
Distribution Of Fatty Acids In Solid Nannochloropsis Pellets

| | Percent (w/w) found |
|---|---|
| Fatty Acid | (as free fatty acid) |
| Saturated fatty acids | 1.4 |
| C16:0 Palmitic acid | 1.3 |
| C18:0 Stearic acid | 0.06 |
| Monounsaturated fatty acids | 0.8 |
| C16:1 Palmitoleic acid | 0.4 |
| C18:1, n-9 Oleic acid | 0.2 |
| C18:1 Octadecanoic acid | 0.04 |
| Polyunsaturated fatty acids | 2.7 |
| C18:2, n-6 Linoleic acid | 0.8 |
| C18:3, n-3 alpha-Linolenic acid | 1.2 |
| C20:4, n-6 Arachiodonic acid | 0.1 |
| C20:5, n-3 Eicosapentaenoic acid | 0.6 |
| Unknown fatty acids | 1.2 |

### Determination of Free Oil Content In Solid Nannochloropsis Pellets

Free oil is normally determined by stirring a sample with n-heptane, centrifuging, and then evaporating the supernatant to dryness. The resulting residual oil is then determined gravimetrically and expressed as a weight percentage of the original sample. This procedure was not found to be satisfactory for the pelletized algae sample, because the resulting residue contained significant levels of pigments. Thus, the procedure above was modified by collecting the residue as above, adding the C15:0 internal standard in known amount, and then analyzing by GC/FID using the same parameters as for total oil determination. In this way, the free oil content of the sample was determined to be 3.7%. After normalization, the free oil content in the algae was determined to be 4.5% (i.e., 3.7% divided by 0.821).

### SCF Extraction of Solid Nannochloropsis Pellets

The extraction vessel was charged with 24.60 g of solid pellets (on a dry weight basis), resulting in about 21.24 g of algae on correcting for the grinding and binding agents. The pellets were flushed with CO₂, then heated to about 40° C. and pressurized to approximately 311 bar. The pellets were extracted at these conditions at a flow rate of 3.8 g/min CO₂ for about 6.7 hr, giving a final solvent-to-feed (S/F) ratio of approximately 71 g CO₂/g algae. The extraction yield was 6.2% of the charged algae.

Based on the above, it is concluded that the process described herein [i.e., comprising steps of (a) mixing a microbial biomass, having a moisture level and comprising oil-containing microbes, and at least one grinding agent capable of absorbing oil, to provide a disrupted biomass mix; (b) blending at least one binding agent with said disrupted biomass mix to provide a fixable mix capable of forming a solid pellet; and (c) forming said solid pellet from the fixable mix] can be successfully utilized to produce solid pellets comprising disrupted microbial biomass from Nannochloropsis. It is hypothesized that the methodology will prove suitable for numerous other oil-containing microbes, although it is expected that optimization of the process for each particular microbe will lead to increased disruption efficiencies. Furthermore, the present Example demonstrates that the solid Nannochloropsis pellets can be extracted with a solvent to provide an extract comprising the oil, in a variety of means. As is well known in the art, different extraction methods will result in different amounts of extracted oil; it is expected the extraction yields may be increased for a particular solid pellet upon optimization of the extraction process.

We claim:

1. A process comprising:
a) mixing a microbial biomass, having a moisture level and comprising oil-containing microbes, and at least one grinding agent capable of absorbing oil, to provide a disrupted biomass mix;
b) blending at least one binding agent with said disrupted biomass mix to provide a fixable mix capable of forming a solid pellet; and,
c) forming said solid pellet from the fixable mix.

2. The process of claim 1 wherein said at least one grinding agent has a property selected from the group consisting of:
a) said at least one grinding agent is a particle having a Moh hardness of 2.0 to 6.0 and an oil absorption coefficient of 0.8 or higher as determined according to ASTM Method D1483-60;
b) said at least one grinding agent is selected from the group consisting of silica and silicate; and,
c) said at least one grinding agent is present at about 1 to 20 weight percent, based on the summation of the weight of microbial biomass, grinding agent and binding agent in the solid pellet.

3. The process of claim 1 wherein the moisture level of the microbial biomass is in the range of about 1 to 10 weight percent.

4. The process of claim 1 wherein said at least one binding agent has a property selected from the group consisting of:
a) said at least one binding agent is selected from water and carbohydrates selected from the group consisting of: sucrose, lactose, fructose, glucose, and soluble starch; and,
b) said at least one binding agent is present at about 0.5 to 10 weight percent, based on the summation of the weight of microbial biomass, grinding agent and binding agent in the solid pellet.

5. The process of claim 1 wherein steps (a) mixing said biomass and (b) blending at least one binding agent are performed in an extruder, are performed simultaneously, or are performed simultaneously in an extruder.

6. The process of claim 1 wherein step (c) forming said solid pellet from said fixable mix comprises a step selected from the group consisting of:
(i) extruding said fixable mix through a die to form strands;
(ii) drying and breaking said strands; and,
(iii) combinations of step (i) extruding said fixable mix through a die to form strands and step (ii) drying and breaking said strands.

7. The process of claim 1 wherein said solid pellets have an average diameter of about 0.5 to about 1.5 mm and an average length of about 2.0 to about 8.0 mm.

8. The process of claim 1 wherein the solid pellets are formed using a granulator, are dried using a fluid bed dryer, or are formed using a granulator and are dried using a fluid bed dryer.

9. The process of claim 1 wherein the oil-containing microbes are selected from the group consisting of yeast, algae, fungi, bacteria, euglenoids, stramenopiles and oomycetes.

10. The process of claim 1 wherein the oil-containing microbes comprise at least one polyunsaturated fatty acid in the oil.

11. The process of claim 1 wherein the microbial biomass is a disrupted biomass, having a disruption efficiency of at least 50% of the oil-containing microbes.

12. The process of claim 11 wherein the microbial biomass is disrupted to produce a disrupted biomass in a twin screw extruder comprising:
(a) a total specific energy input (SEI) of about 0.04 to 0.4 KW/(kg/hr);
(b) compaction zone using bushing elements with progressively shorter pitch length; and,
(c) a compression zone using flow restriction;
wherein the compaction zone is prior to the compression zone within the extruder.

13. The method of claim 11 wherein said flow restriction is provided by reverse screw elements, restriction/blister ring elements or kneading elements.

14. The process of claim 11, further comprising:
d) extracting the solid pellet with a solvent to provide an extract comprising the oil.

15. The process of claim 12, wherein the solvent comprises liquid or supercritical fluid carbon dioxide.

16. A pelletized oil-containing microbial biomass made by the process of claim 1.

17. A solid pellet comprising:
a) about 70 to about 98.5 weight percent of disrupted biomass comprising oil-containing microbes;
b) about 1 to about 20 weight percent of at least one grinding agent capable of absorbing oil; and,
c) about 0.5 to 10 weight percent of at least one binding agent; wherein the weight percents of (a), (b) and (c) are based on the summation of (a), (b) and (c) in the solid pellet.

18. The solid pellet of claim 17 wherein said pellets have a property selected from the group consisting of:
(a) an average diameter of about 0.5 to about 1.5 mm and an average length of about 2.0 to about 8.0 mm; and,
(b) a moisture level of about 0.1 to 5.0 weight percent.
